(19) United States
(12) Patent Application Publication
(10) Pub. No.: US 2007/0272010 A1
(43) Pub. Date: Nov. 29, 2007

(54) Assessment Apparatus and Method

(75) Inventors: Shaun Patrick O'Leary, St Lucia (AU); Gwendden Anne Jull, The Gap (AU); Guglisimo Tarcisio Vicenzino, Ashgrove (AU); Matthew Campbell Greaves, St Lucia (AU)

Correspondence Address: DENNISON, SCHULTZ & MACDONALD, 1727 KING STREET, SUITE 105, ALEXANDRIA, VA, 22314, US

(73) Assignee: The university of Queensland

(21) Appl. No.: 10/572,454

(22) PCT Filed: Sep. 20, 2004
(86) PCT No.: PCT/AU04/01279

§ 371 Date: Jul. 25, 2007

(30) Foreign Application Priority Data
Sep. 19, 2003 (AU) 2003905108

(51) Int. Cl. A61B 5/22

(52) U.S. Cl. 73/379.01

(57) ABSTRACT

A method and apparatus are described for assessing function of the cranio-cervical muscles. The method includes assessment of torque produced during flexion/extension, axial rotation, lateral rotation or combination thereof. The apparatus (110) has a lever arm (130) mounted to a support frame (120), wherein the length of the arm and its angle of extension may be varied. Torque is assessed at an anatomical axis of rotation in a subject. The apparatus axis of rotation may be adjustable. A preferred embodiment has a pivoted support frame for positioning over a subject's head, wherein the support frame (120) may form a lever arm for rotation. The apparatus may be removably mountable to an upright such as a door jamb.

### FIELD OF THE INVENTION

THIS INVENTION relates to a device and method for assessing the performance of a muscle or muscles in a subject. More particularly, the invention relates to a device and method for assessing the cranio-cervical muscles of a subject but is not limited to this group and may be applied to other muscles and/or muscle groups.

### BACKGROUND OF THE INVENTION

Neck pain affects 13-20% of the population [1, 2] and approximately 70% of all people will experience neck pain at some point in their life [2, 3]. Traumatic neck injury such as whiplash, is the most commonly reported injury in some jurisdictions. In addition 16% of the population has a headache at any one time [5] and 14-18% of these headaches originate from the neck (cervicogenic headache) [6, 7]. Neck pain and back pain are the most common reasons for visits to a physiotherapist (42% of visits) [8]. In recent years substantial evidence has emerged identifying impairment in the muscle system which invariably accompany chronic neck pain.

In particular, impairment has been demonstrated in those muscles that control motion of the head on the neck, the cranio-cervical muscles (“C-C muscles”). Deficits have been demonstrated in the C-C flexor [9-13], extensor [14-17], and rotator muscles [17-20] and include: a loss of strength and endurance [10, 11, 13, 21, 22], poor coordination and efficiency between muscles within a group [11, 23, 24], muscle wasting and changes in muscle composition [17], a reduction in the way the C-C muscles control the head for vision and balance function [18, 20, 21]. These muscle impairments have been demonstrated in both idiopathic [10, 13, 22, 24] and traumatic [11, 18] neck conditions. Additionally a randomised clinical trial demonstrated that rehabilitation of the C-C flexor muscles was successful in providing long term relief of neck pain and headache [12]. There is however no direct method to measure the performance of these muscles, which hampers scrutiny of their involvement in neck pain and limits the clinical implementation and assessment of a structured exercise program. The only method of measurement of C-C muscle performance that could also be used for graded rehabilitation of the C-C muscles is the pressure biofeedback unit (PBU) (Chattanooga, USA). The PBU is however an indirect measure, and can only be used for C-C flexion. It would be of advantage to provide a device and method to assist in assessing the function of at least the C-C muscles.

Reference to any prior art in this specification is not, and should not be taken as, an acknowledgment or any form of suggestion that this prior art forms part of the common general knowledge in any country.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In a first aspect, the invention resides in an apparatus for assessing muscle performance in a subject, the apparatus comprising:

a force receiving member having a subject input region adapted to receive force input from the subject;

an axis of rotation of the force receiving member spaced from the input region; and

torque assessment means for determining or assessing torque produced at the axis of rotation of the force receiving member by the force input from the subject.

In this regard, “torque” is the product of a force and its perpendicular distance from a point about which it causes rotation or torsion.

The apparatus may be adapted for assessment of performance of the cranio-cervical muscles. However, in some embodiments, other muscles or muscle groups may be suitable for assessment with the present apparatus.

The muscle performance of the cranio-cervical muscles may include one or more of flexion, extension, axial rotation, lateral flexion or any combination thereof.

The force receiving member is preferably a force receiving arm and may be a lever arm. The lever arm may be formed as a right angled member although any suitable shape can be utilised. The right angled member may have an arm portion and an offset subject engaging portion forming or including the subject input region. The subject input region may be padded or, alternatively or additionally, may have a chin seat for receiving the chin of the subject. The chin seat may be rotatably disposed on the offset portion. The subject input region may be adapted to accommodate any suitable anatomical structure such as the jaw, head, chin or other physical feature.

In a preferred embodiment, the length of the arm portion between the axis of rotation and the subject input region is variable as is the angle at which the lever arm extends.

The apparatus may include a support frame for supporting the lever arm. The lever arm may be attached to the support frame by locking means preferably at or around the axis of rotation and adapted for variation of the length and angle of the lever arm. The locking means may be adapted for lockably engaging the lever arm relative to the support frame. The locking means may comprise a bore with a locking arrangement adapted to lock or release the lever arm to thereby permit the lever arm to slide back and forth in the bore. The locking arrangement may be a grub screw, quick release lever, pin or similar. The lever arm may be telescopic. A skilled addressee will be aware of many variations to the locking arrangement that would be suitable for this function.

Torque assessment means may be adapted for determining force applied to the subject input region and distance from the subject input region to the axis of rotation for calculating or assessing torque produced at the axis of rotation. Torque assessment means may include force determining means. The lever arm may be operatively engaged with force determining means. Force determining means may include a dynamometer torque arm. The dynamometer torque arm may be rigid and may abut a torque load cell for determining the force applied to the subject input region. The load cell may be in a form as is well known to a person skilled in the field. Force determining means may comprise a rigid dynamometer torque arm, a rotatable dynamometer torque arm and a load cell there between, wherein force applied to the subject input region will provoke activity in the torque load cell by movement or tendency to movement between the rigid and moveable torque arm. The force determining means may comprise a pneumatic or fluid arrangement with one or more indicator gauges to assess force applied and/or torque at the axis of rotation. The pneumatic or fluid arrangement may be fixed to an arm which, in turn, is fixed to the axis of rotation. Torque may be calculated by multiplying the force by the known length of the arm. Optionally, a spring force detection arrangement may be used to assess force and/or torque in similar manner to the pneumatic or fluid arrangement. Torque may be measured directly be an appropriate sensor or other arrangement.

The support frame may include a frame member adapted for location around a subject's head. The support frame may include location members for positioning on either side of the subject's head. The location members may be formed as ear pads. The ear pads may be rotatably mounted to the frame member to allow rotation of the subject's head relative to the frame member, conveniently in the sagittal plane. This is particularly suitable for flexion and the extension of the C-C muscles. The ear pads can be adjustable to vary the space between them. The ear pads may be operable by quick release levers to allow the ear pads to be advanced or retracted relative to each other. A lever arm for positioning at or around a subject's chin may be pivoted on an axis substantially between the centres of the ear pads which may be adapted to align the point of rotation of the lever arm with or around the subject's axis of rotation when conducting C-C flexion and extension.

The frame member is preferably U-shaped. The frame member may be dorsally pivoted to provide a second lever arm in the apparatus and to permit rotation of the subject's head in the transverse plane. The ear pads may form the subject input region in this application. The frame member may include brake means to allow the frame member to be adjusted between rotatable and fixed. The frame member may be adjustable to different positions within its arc of rotation to allow assessment of C-C rotational or flexion/extension function at different positions in the arc of rotation of a subject's head.

The brake means may include a quick release lever activating a cammed locking arrangement to deform a shaft to lock on a surrounding through bore.

The support frame may further comprise mounting means for fixing to a support structure. The mounting means may comprise a clamping arrangement. The clamping arrangement may comprise two opposed jaws adjustable to be advanced towards or retracted form each other to facilitate mounting of the device to an upright structure such as a post or, preferably, a doorjamb. The jaws may be adjusted by one or more screw threaded shafts mounted to move the jaws.

The apparatus may comprise travel control means to allow controlled rotation of the lever arm. The travel control means may include activation means such as a motor to rotate the lever arm. Speed governing means may also be included to allow the rate of rotation to be set and preferably varied.

Travel control means may provide a substantially constant resistance to movement. Travel control means may comprise a suspended load positioned to resist movement. The load may be variable. Travel control means may comprise one or more springs arranged to resist travel of the lever arm, preferably constantly.

Alternatively, the travel control means may comprise one or more piston arrangements, wherein a piston is mounted slidably in a cylinder, the cylinder containing a fluid which may be pressured to resist inward movement of the piston. The piston may, in turn, be connected directly or indirectly to a lever arm. Pressure of the fluid may be variable. The fluid may be liquid but is preferably a gas.

The piston arrangement may be connected to the lever arm by a cable. The cable is preferably a bowden-type cable which comprises a flexible cable used to transmit mechanical force or energy by movement of an inner cable relative to a hollow outer cable. The cable may be attached to the lever arm at or around the axis of rotation of the lever arm or to an attachment extending therefrom. Preferably, the cable is attached to an indicator arm attached for rotation around the AOR of the device. Most preferably, the cable is fixed so as to activate the piston in either direction of rotation.

Preferably, the device has two piston arrangements, one adapted for flexion extension of the C-C musculature and the other adapted for rotation of the C-C musculature. A third piston arrangement may be provided for lateral C-C flexion.

A switch arrangement may be provided to prevent operation of one or other of the pistons at any one time. The switch arrangement may comprise a lockout rod.

The apparatus may include adjustment means for altering the position of the axis of rotation of the lever arm relative to the support frame and a base of the apparatus, preferably a base plate.

The adjustment means may suitably comprise either or both vertical and horizontal adjustment means. The vertical and horizontal adjustment means may comprise vertical and horizontal rods which are moveable relative to each other and clamping means for fixing the vertical and horizontal rods in relative position or releasing them for movement. The vertical rod or rods may be fixed to the base plate. The adjustment means may include the capacity to orientate the lever arm for operation in the saggital, coronal and transverse planes, respectively. The adjustment means may include separate, alternative fixing points for the lever arm on the support frame and/or the base plate.

The apparatus may further comprise subject head support means. The subject head support means may be adapted to support the head of the subject during assessment. The head support means may be slidable, preferably with low friction. The head support means may include a force determining means for determining the amount of force applied to the head support means by the subject. The force determining means may be a force transducer. The force transducer may be in so connection with means for determining, displaying and/or recording force applied to the head support means by the subject's head. The head support means may be adapted to support both the head and neck of a subject.

The apparatus may further comprise a subject support platform. The subject support platform may be a plinth. The subject support platform may include a harness or harnesses for supporting the legs of the subject. The subject support platform may include a strap or straps for supporting or restraining the torso of a subject. Alternatively or additionally, the subject support platform may be adapted to support a subject in a sitting position. The subject support platform may be substantially in the form of a chair.

The apparatus may further comprise amplifying mews for amplifying voltage changes detected by measuring devices in the apparatus. The measuring devices may be the force transducer or torque load cell and/or the force load cell for the head.

The apparatus may further comprise processing means for receiving signal input from the force measuring components of the apparatus and preferably displaying and analysing data relating to the subject's muscle performance.

The processing means may be a computer. The computer may be programmed to determine torque by applying the algorithm:
T=F×D;

where T=torque, preferably in newton-metres;

F=fore, preferably in newtons, applied by the subject;

D=distance, preferably in metres, between the subject input region and the axis of rotation, the distance being perpendicular from the axis of rotation.

Alternatively, F may be the force indicated by force determining means and D may be the length of the indicator arm.

Alternatively, the computer may be programmed to record force (preferably in newtons) or torque when the piston arrangement is connected at or around the axis of rotation.

The computer may be further programmed to provide an indication of function of the muscle performance of the subject. The indication may be in a range such as poor, average, excellent. In an alternative embodiment, data from assessment of a subject may be recorded on an electronic, transportable recording medium such as a “smart card” or a “floppy disk”, for subsequent presentation to processing means and/or storage.

The apparatus may further comprise display means for providing feedback to a subject. The display means may be an audible signal and/or a visual signal such as a visual display unit. The visual display unit may be adjustable such that it can be positioned in full view of the patient or removed from the view of the patient if desired. The visual display unit can be programmed to display only parts or all of the test outcomes both during and following the completion of the test eg. Torque, head weight force, or other information used in the test such as electromyographic information as described below. The visual display means may comprise a pressure, force or torque gauge for also measuring pressure or force generated by a subject in performing an exercise, particularly an isometric exercise. The gauge may be a hydraulic gauge having an input line co-operatively coupled to a corresponding piston arrangement, wherein the line may be in fluid connection with the gauge when the corresponding piston arrangement is locked to thereby prevent movement of the piston and lever arm and provide an isometric application for the subject. The apparatus may comprise two gauges, each connected to a corresponding piston arrangement which may be for flexion/extension in the sagittal plane and rotation in the transverse plane, respectively. The gauges may provide an indication of isometrically provided force. In an alternative embodiment, the gauges may be electronic gauges as are well known. The gauges may be in signal connection with a display such as an LED display and also with processing means such as a computer for recording and/or analysis of the results.

The apparatus may also include electromyographic monitoring means for monitoring the electrical activity of muscles under review and/or additional muscles when performing muscle tests of varying contraction intensities. This may be used in addition to the torque and head weight force information for diagnosis or assessment of muscle impairment and feedback during rehabilitation.

In a further aspect, the invention resides in a dynamometer or apparatus for assessing muscular generation of torque, the dynamometer comprising:

one or more lever arms rotatably mounted to a support frame and preferably each having contact padding arranged on a subject contact region;

torque measuring or assessing means;

locking means for locking the lever arm in position relative to an axis of rotation of the apparatus;

wherein the length of the lever arm and its angle to the mounting point are variable.

The torque measuring or assessing means may comprise force measurement means adapted to measure or assess force applied to the subject contact region.

The dynamometer may include adjustment means for adjusting the axis of rotation of the lever arm relative to the support frame and/or a base of the dynamometer.

adjustment means for adjusting the axis of rotation relative to the support frame and/or a base of the dynamometer.

Preferably, the dynamometer also includes:

a head support platform supported by the base and adapted to receive the head of the subject; and

a load cell for determining the force of the head on the head support platform.

The dynamometer may have two or three separate mounting points or hubs for receiving the lever arm in alternative positions the lever arm.

The lever arm distance indicator may be automated. The lever arm bolt may be arranged to release the lever arm for variation of the distance between the axis of rotation and the patient contact region.

The head platform may be supported on ball bearings for low friction sliding.

In yet a further aspect, the invention resides in a method of assessing muscular function, the method comprising the steps of:

determining or obtaining an indication of the torque produced by a muscle or a group of muscles.

The method may ether comprise the steps of;

mounting a lever arm on a support base or support frame;

positioning the lever arm in contact with an anatomical structure activated by the muscle or muscles;

activating the muscle or muscle groups of the subject; and

determining the force produced by the muscle or muscles; and

calculating or assessing torque produced by the muscle or muscles.

Preferably the muscles are the cranio-cervical group of muscles. Activating the muscles may include one or more of flexion, extension, axial rotation, lateral flexion or combination thereof.

The method may also comprise locating an axis of rotation of a lever arm substantially coincident with an axis of rotation of the anatomical structure. This step may include identifying the axis of rotation of the anatomical structure.

Preferably, the method includes adjusting the lever arm so that it engages the anatomical structure at a predetermined site and determining the perpendicular distance from the predetermined site to the axis of rotation of the lever arm.

The method may further comprise lateral and vertical alteration of the axis of rotation of the lever arm to be substantially coincident with the axis of rotation of the anatomical structure.

The method may further comprise positioning the lever arm in two or more different mounting positions to enable assessment of different muscular activities such as flexion/extension, axial rotation or lateral flexion. Preferably, three mounting positions are provided, one for each muscular activity.

The method may further comprise assessing the force exerted by one or more additional muscles of the subject which are not subject to the assessment. The one or more additional muscles may cause increased or decreased pressure through the head of the subject. The method may comprise monitoring the downward, upward, or sideways pressure of the subjects head with a force transducer. Alternatively, or additionally, the method may comprise electromyographic monitoring of the additional muscles to identify inappropriate recruitment of those muscles during prescribed exercises.

The method further comprises analysing the results of the subject's assessment to provide an indicator of muscular function. That indicator may be in a range such as poor, good or excellent. The method may further comprise comparing the results of a subject's performance with prior results for the same subject and/or against a collected database of results of subjects. The method may include comparing performance between two or more C-C muscle groups. The method may include comparing muscle function of C-C muscle groups with the larger cervical muscles. The method may comprise entry of the results of a subject's performance into a database to contribute to, compare with, or develop a reference database.

The method may further include automatically determining the distance from the axis of rotation of the lever arm to the patient contact region.

The method may also include suspending a subject's legs in a harness or harnesses and/or restraining the torso of the subject in a band, harness or similar. The subject's arms may be restrained under the harness or band, preferably in a crossed position.

The method may include one or more of:

(1) maximal voluntary contraction;

(2) sustained voluntary contraction;

(3) oscillatory voluntary contraction (ie. back and forth);

(4) attaining a prescribed level of torque production; and/or

(5) combination of prescribed levels of torque production variable within a set duration.

In another aspect, the invention resides in a machine readable program adapted to program a machine such as a computer to receive input from force measuring sensors in a device as described above and to determine torque produced by a subject's application of muscle force to the device. The program may be further adapted to record input from a force load cell positioned to determine head force of the patient. The program may be adapted to provide a visual readout of the results and data received. The program may also be adapted to determine the axis of rotation of an anatomical structure. The invention may also reside in a machine programmed with machine readable code as described.

In a further aspect, the invention may reside in a method of rehabilitation of C-C muscle function comprising repeated application of one or more of the above described embodiments of the method.

In yet a further aspect, the invention may reside in a method of treating head and/or neck pain by repeated application of one or more of the above described embodiments of the method.

### BRIEF DESCRIPTION OF THE DRAWINGS

In order to provide a better understanding of the present invention, preferred embodiments will be described in detail, by way of example only, with reference to the accompanying drawings in which:

FIG. 1 shows standard anatomical planes of a subject in which C-C flexion and extension occurs in the sagittal plane, C-C rotation in the transverse plane, and C-C lateral flexion in the coronal plane.

FIG. 2 is a side view of a subject in neutral standing position. C-C Neutral in standing is denoted by a horizontal position of the Frankfort Plane which is parallel to the horizontal plane.

FIG. 3 is a side view of a subject in neutral supine position. C-C Neutral in supine is denoted by a vertical position of the Frankfort Plane which is parallel to the transverse plane

FIG. 4 is a top view of a subject in neutral supine position. C-C Neutral in supine is denoted by the sagittal plane bisecting the body into symmetrical halves

FIG. 5 shows a side view of a subject performing C-C flexion and flexion of the head and neck. The comparison between the movements of C-C flexion (A), and flexion of the head and cervical spine (B) is indicated by the arrows. The locations of the AOR about which the different motions occur is demonstrated by a white marker and indicate why torque may be specific to the AOR of the movement

Panel A. C-C Flexion and AOR location represented by white marker.

Panel B. Flexion of the head and neck and AOR location represented by white marker.

Panel C. Neutral Position.

FIG. 6 is a side view of a subject shown in end range flexion, end range, extension and neutral positions. The panels disclose the following:

A=C-C Neutral

B=End range C-C Flexion

C=End range C-C Extension

Inner Range C-C Flexion—Motion from positions A-B is inner range C-C flexion.

Outer Range C-C Flexion—Motion from positions C-A is outer range C-C flexion.

Full Range C-C Flexion—Motion form positions C-B is full range C-C flexion.

FIG. 7 illustrates flexor muscles of the cranio-cervical region, namely: C-C flexor muscles; Rectus capitis anterior and Longus capitis. The Hyoid muscles are not depicted in this figure. As demonstrated these muscles are attached to the front of the cervical spine and are attached to the skull. Their action is rotate the head on the neck so that the chin approximates the front of the neck.

FIG. 8 shows a subject conducting C-C extension and extension of the head and neck.

The comparison between the movements of C-C extension (A), and extension of the head and cervical spine (B) indicated by the arrows. The locations of the AOR about which these different motions occur is demonstrated by a white marker and indicate why torque may be specific to the AOR of the movement.

Panel A. C-C Extension and AOR location represented by white marker.

Panel B. Extension of the head and neck and AOR location represented by white marker.

Panel C. Neutral Position

FIG. 9 is a side view of a subject shown in end range extension, end range flexion and a neural position.

A=C-C Neutral

B=End range C-C Extension

C=End range C-C Flexion

Inner Range C-C Extension—Motion from positions A-B is inner range C-C extension.

Outer Range C-C Extension—Motion from positions C-A is outer range C-C extension.

Full Range C-C Extension—Motion form positions C-B is full range C-C extension.

FIG. 10 shows short C-C extensor muscles.

Short CC extensor muscles. 1. Rectus capitis posterior major. 2. Rectus capitis posterior minor. 3. Obliquus capitis superior.

FIG. 11 shows long C-C extensor muscles.

Long C-C extensor muscles 1. Semispinalis capitis. 2. Longissimus capitis

FIG. 12 shows a subject in top view performing C-C axial rotation and right head and cervical spine axial rotation.

The comparison is between the movements of C-C rotation (A), and rotation of the head and cervical spine (B) The arrow indicates the direction of motion. The white marker indicates the AOR which is expected to be similar for both motions. Motion is limited to 40 degrees rotation to target C1/2 rotation.

A. Right C-C Rotation

B. Right Head and Cervical spine rotation

C. C-C Neutral

FIG. 13 shows a subject, in top view, in end range axial rotation and neutral positions.

A=C-C Neutral

B=End range right C-C Rotation

C=End range left C-C Rotation

Inner Range C-C Rotation—Motion from positions A-B is inner range C-C rotation.

Outer Range C-C Rotation—Motion from positions C-A is outer range C-C rotation.

Full Range C-C Rotation—Motion from positions C-B is full range C-C rotation.

FIG. 14 shows C-C rotator muscles.

C-C rotator muscles targeting rotation to the C1/2 motion segment are shown as multiple muscles that attach to the skull including many of the C-C flexors and extensors when contracting on one side only will contribute to C-C rotation.

FIG. 15 shows a subject performing right C-C lateral flexion and right head and cervical spine lateral flexion.

The comparison is between the movements of right C-C lateral flexion (A), and right lateral flexion of the head and cervical spine (B). The arrow indicates the direction of movement.

A. Right C-C lateral flexion

B. Right head and cervical spine lateral flexion

C. Neutral Position

FIG. 16 shows C-C lateral flexor muscles.

C-C lateral flexor muscles targeting lateral flexion to the C0/1 motion segment are shown as: 1. Rectus capitis posterior major; 2. Rectus capitis posterior minor; 3. Obliquus capitis superior. Multiple muscles that attach to the skull include many of the C-C flexors and extensors which, when contracting on one side only, will contribute to C-C lateral flexion.

FIG. 17 shows a subject in position with an apparatus of the present invention.

C-C Flexion Torque Measurement has the following feature:

A. Dynamometer axis is aligned to the patients AOR.

B. Dynamometer lever arm is extended at a known distance from the axis.

C. When performing C-C flexion the patients chin exerts a force on the dynamometer lever arm. The torque is the product of the force (newtons) produced by the chin and the length of the lever arm (meters).

FIG. 18 shows steps in the process of acquiring torque data.

Flow Chart for Acquisition of Torque Data.

FIG. 19 shows steps in the process of acquiring head force data and includes a flow chart for acquisition of head force data.

FIG. 20 shows graph results from an apparatus of the present invention. Traces of the torque output and the head weight force output during three repeated trials of C-C flexion maximal voluntary contractions. These outputs can be recorded visually in this form or in continuous values in spreadsheet form.

FIG. 21 shows results of an endurance task using the present invention. Traces of the torque output and the head weight force output during an endurance task of C-C flexion (20% of MVC) for 60 seconds. These outputs can be recorded visually in this form or in continuous values in spreadsheet form.

FIG. 22 shows the set up for dynamometric testing in extension using an apparatus of the present invention.

C-C Dynamometry set-up for the measurement of C-C extension torque

FIG. 23 shows the apparatus of the present invention when positioned for C-C axial rotational torque and is an example of C-C Dynamometry lever arm set-up for the measurement of right C-C rotation torque. The lever arm is preferably attached to the base and support frame (seen to the right hand side of the head) suitably positioned so this axis is aligned to the vertex of the head indicated by the arrow.

FIG. 24 shows arrangement for determining C-C lateral flexion torque.

An example of C-C Dynamometry lever arm set-up for the measurement of left C-C lateral flexion torque. The lever arm is preferably attached to the base (seen to the right hand side of the head) suitably positioned so the axis is aligned to the head indicated by the arrow.

FIG. 25 shows a subject in position with a visual display feedback arrangement.

An example of CC dynamometry using a visual feedback display. The screen above the patient's bead displays the measurement of torque and/or head weight force for measurement and retraining purposes.

FIG. 26 is a first side view of a first embodiment of a dynamometer of the present invention.

FIG. 27 is a reverse view to that of FIG. 26.

FIG. 28 is a front view of an alternative embodiment of an apparatus of the present invention in which a subject is seated.

FIG. 29 is a side view of the apparatus of FIG. 28.

FIG. 30 is a perspective view of a further alternative embodiment of an apparatus of the present invention.

FIG. 31 is a front view of the embodiment of FIG. 30.

FIG. 32 is a side view of the embodiment of FIG. 31 also showing hydraulic lines connected to gauges.

FIG. 33 is a top view of the arrangement of FIG. 32.

FIG. 34 is a perspective view of two piston arrangements for use with an apparatus of the present invention and particularly suitable for the embodiment of FIG. 30.

FIG. 35 is a sectional view of the arrangement of FIG. 34.

FIGS. 36 to 38 show one arrangement for attachment of a bowden cable for two-way operation.

### DETAILED DESCRIPTION OF THE DRAWINGS

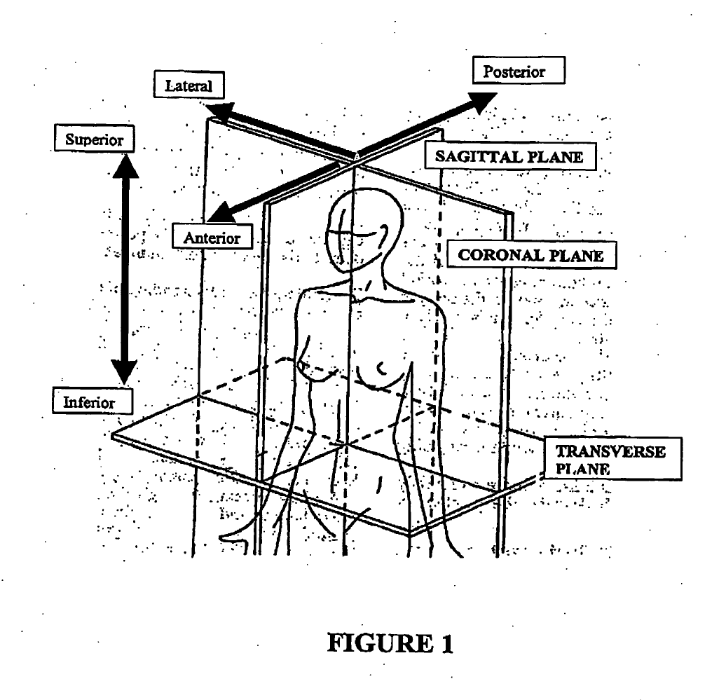

The Cranio-Cervical (“C-C”) region is that region where the bony head joins onto the bony neck and its associated soft tissues such as muscle, ligament, and nerve tissue. For the purposes of this specification the skull and mandible will be described as a single unit and referred to as the head. C-C motion describes motion of the head on the neck and is primarily produced and controlled by the muscles that attach the spine directly to the head. The principle anatomical directions of C-C spine motion are C-C flexion, extension, axial rotation, and lateral flexion however infinite combinations of these movements may occur. Anatomical motions may be described in terms of the anatomical planes of motion along which the motion occurs. The planes of motion are illustrated in FIG. 1. C-C flexion and extension occurs in the sagittal plane, C-C axial rotation occurs in the transverse plane and C-C lateral flexion occurs in the coronal plane.

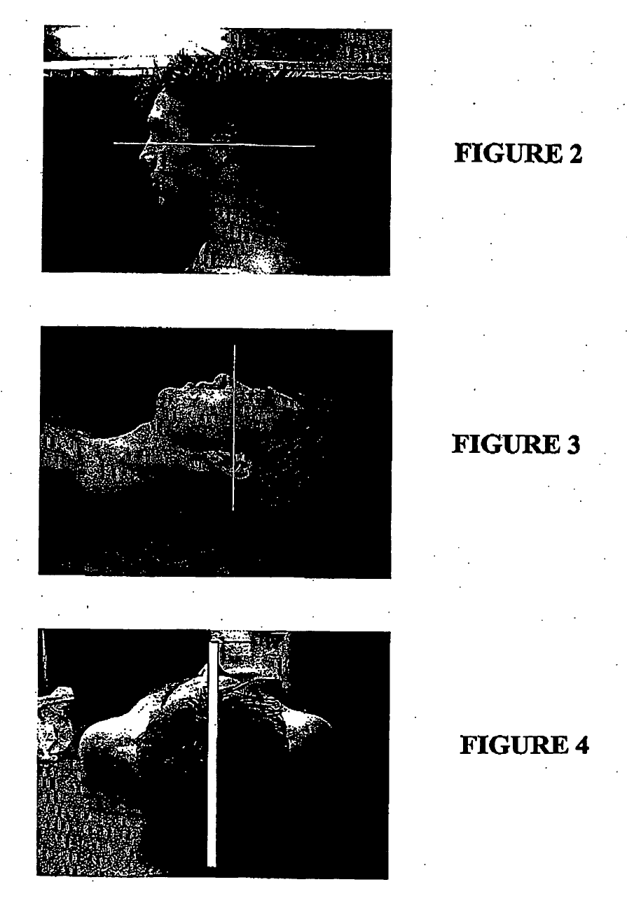

In order to describe motion of the head on the neck, a neutral stating position should be defined C-C neutral is a position in which the bead is neither flexed, extended, rotated, laterally flexed, or a combination of any of these positions in relationship to the neck. Due to the difficulty in locating the neutral position of the C-C articulations non-invasively, a standard anthropometric neutral position will be used, the Frankfort Plane [25]. The Frankfort Plane describes the position of the head in relationship to the anatomical planes and is defined by a line joining the anatomical landmarks of the orbitale (lower edge of the eye socket) and the tragion (the notch superior to the tragus of the ear) as shown in FIG. 2. In a neutral position the Frankfort Plane is parallel to the anatomical transverse plane (see FIG. 1). Therefore when an individual is in a standing position with the spine in an upright position the Frankfort Plane should be horizontal, if the individual is lying down on their back in a horizontal position Frankfort Plane should be vertical (see FIG. 3). In this position the C-C spine is neither flexed or extended. C-C neutral will also require that the sagittal plane should symmetrically bisect the head and neck into halves so that the head is not in axial rotation or is not laterally flexed (see FIG. 4).

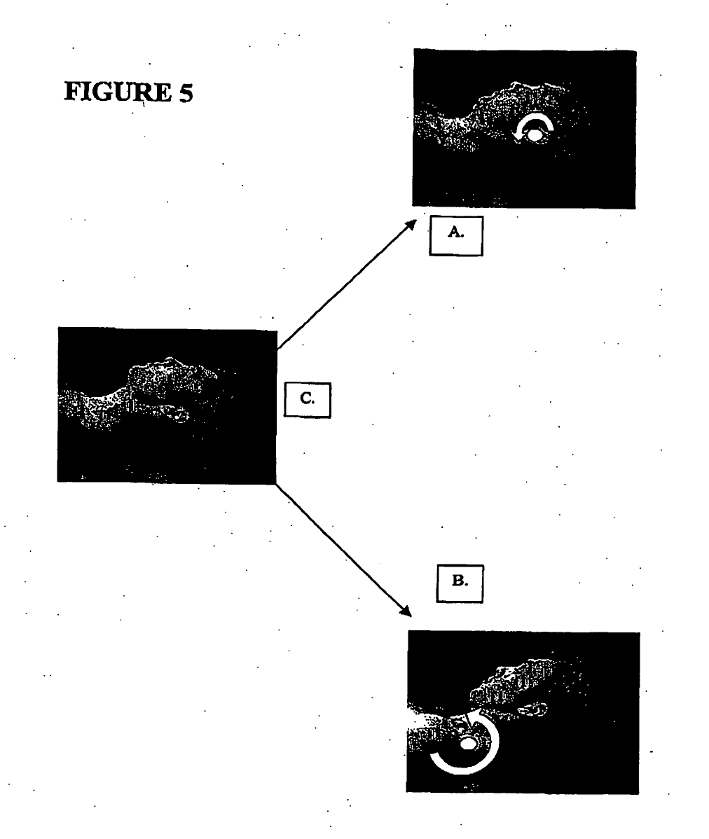

C-C Flexion describes rotational motion of the head on the neck in the sagittal plane such that the chin approximates the front of the neck (nodding type movement). This rotational motion of the head should occur primarily at the atlanto-occipital (C0/1) articulation about it's axis of rotation (AOR) as shown in FIG. 5. Theoretically this AOR landmark should not translate anteriorly or posteriorly, instead should be a rotation point. This AOR differs from that of flexion of the head and neck which occurs about an AOR around the articulation of the seventh cervical (C7) and first thoracic spine vertebrae (T1) as illustrated in FIG. 5 and is associated with a large excursion of anterior translation of the head anteriorly in the sagittal plane.

Despite the primary articulation of C-C flexion being C0/1 motion there will be some motion into flexion of the other cervical motion segments due to the associated effects of muscle contraction. Rotational motion of the head will therefore also be accompanied by a reduction in the cervical lordosis.

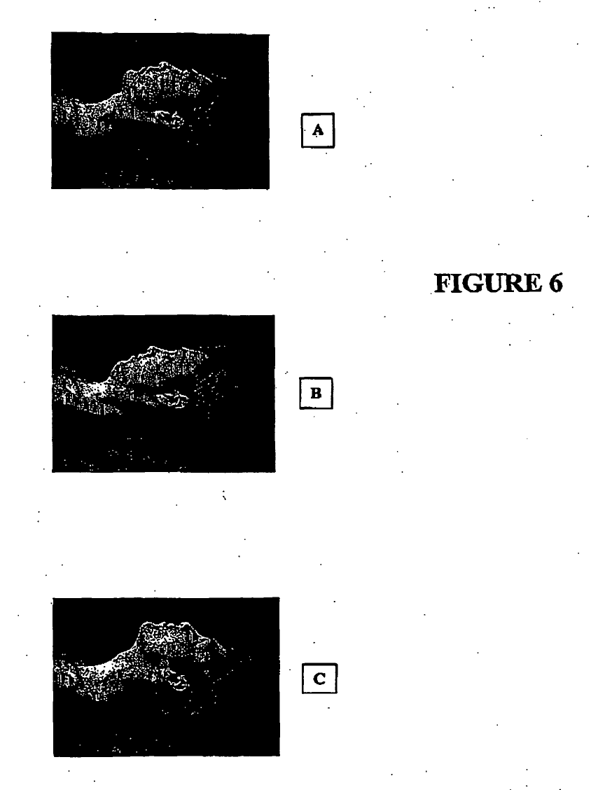

The term C-C flexion can be used to describe both a static position of the C-C region and/or a direction of C-C motion. When the C-C region is described as being in C-C flexion (or flexed), this means it will be in a position between the neutral C-C position and the end of range of C-C flexion (see FIG. 6), C-C flexion when being described as a direction of C-C motion may occur in any range of the sagittal plane C-C motion. This motion when performed from the neutral to the end of C-C flexion range is ‘inner range’ C-C flexion. C-C flexion may also be initiated from a position at end range C-C extension as demonstrated in FIG. 6, the motion occurring from end range C-C extension to neutral is called ‘outer range’ C-C flexion. C-C flexion that is initiated end range C-C extension and is finished at end range C-C flexion is called ‘full range’ C-C flexion, C-C flexion motion may be initiated or terminated at any point of the sagittal plane C-C range.

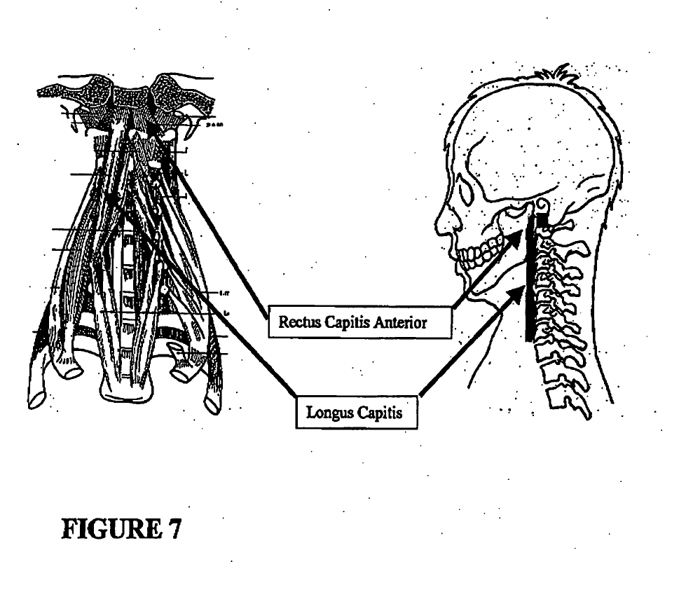

C-C flexion is produced by a group of muscles called the C-C Flexor Muscles as shown in FIG. 7. The primary muscles producing C-C flexion are the: longus capitus, rectus capitus anterior, and the collective hyoid muscles. These muscles all originate from the cervical spine except the hyoid muscles which originate from the sternum, clavicle, and scapula. The C-C flexor muscles attach to the head (skull and jaw) at various distances anterior to the C0/1 articulation and its AOR. The distance the individual muscles are attached from the AOR will determine their lever arm by which they can exert torque at the AOR. Contraction of the C-C flexor muscles therefore exerts a force on their bony head attachment, which results in a torque about the AOR via the lever arm which results in the rotation of the head in the sagittal plane. Torque about this AOR may be produced by the collective contractions of all the C-C flexor muscles.

Muscles which may assist in C-C flexion but can not directly contribute to the motion as they do not attach to the head include: longus cervicis, anterior scalene. Sternocleidomastoid may also assist in the inner ranges of C-C flexion. These muscles assist via their synergistic effects on the cervical spine in support of the C-C flexor muscles.

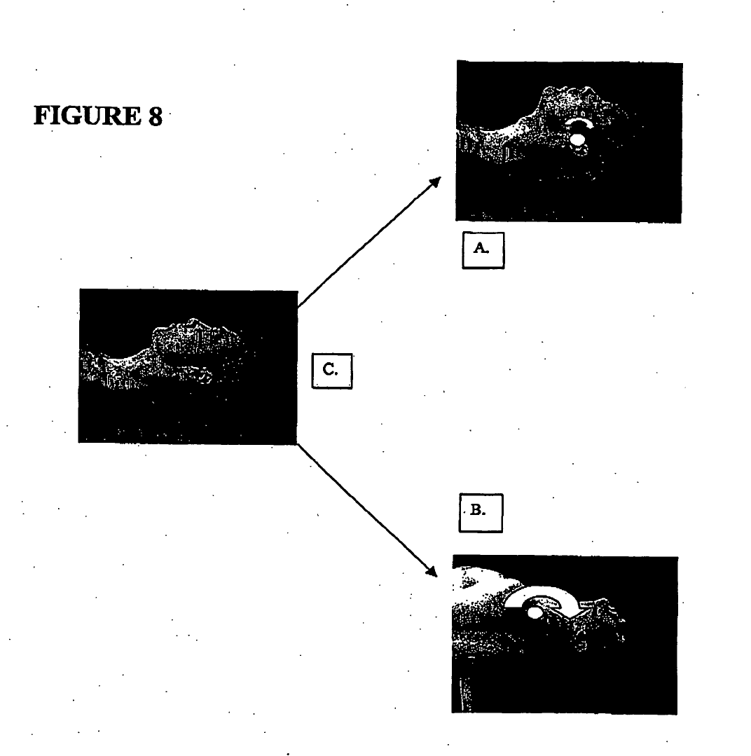

C-C extension describes rotational motion of the head on the neck in the sagittal plane such that the chin separates from the front of the neck. This rotational motion of the head should occur primarily at the atlanto-occipital (C0/1) articulation about it's AOR and is illustrated in FIG. 8. Theoretically this AOR landmark should not translate anteriorly or posteriorly, and instead should be a rotation point. This AOR differs from that of extension of the head and neck which occurs about an AOR around the articulation of the seventh cervical (C7) and first thoracic spine vertebrae (T1) which is also shown in FIG. 8 and is associated with a large excursion of posterior translation of the head posteriorly in the sagittal plane.

Despite the primary articulation of C-C extension being C0/1 motion there will be some motion into extension of the other cervical motion segments due to the associated effects of muscle contraction. Rotational motion of the head will therefore also be accompanied by an increase in the cervical lordosis.

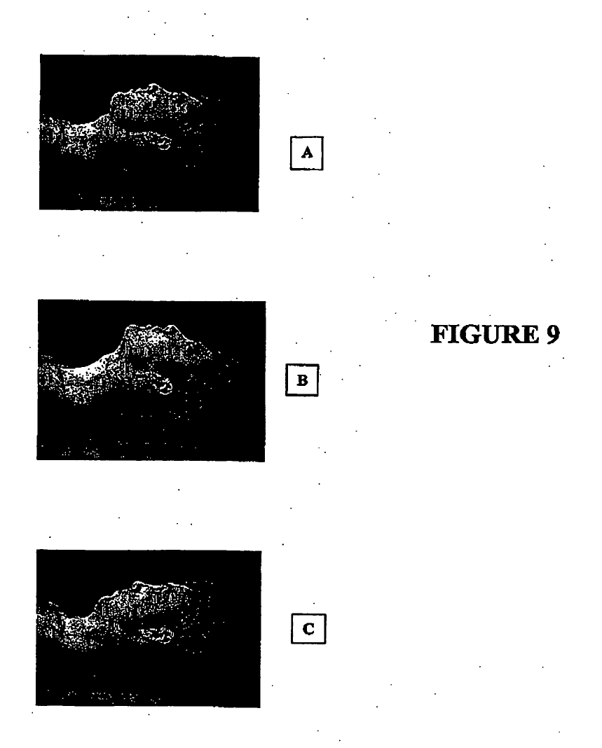

It should be noted that the term C-C extension can be used to describe both a static position of the C-C region and/or a direction of C-C motion. When the C-C region is described as being in C-C extension (or extended), this means it is in a position between the neutral C-C position and the end of range of C-C extension as demonstrated in FIG. 9. C-C extension when being described as a direction of C-C motion may occur in any range of sagittal plane C-C motion. This motion when performed from the neutral to the end of C-C extension range is ‘inner range’ C-C extension. C-C extension may also be initiated from a position at end range C-C flexion, the motion occurring from end range C-C flexion to neural is called ‘outer range’ C-C extension. C-C extension that is initiated at end range C-C flexion and is finished at end range C-C extension is called ‘full range’ C-C extension. C-C extension motion may be initiated or terminated at any point of the sagittal plane C-C range.

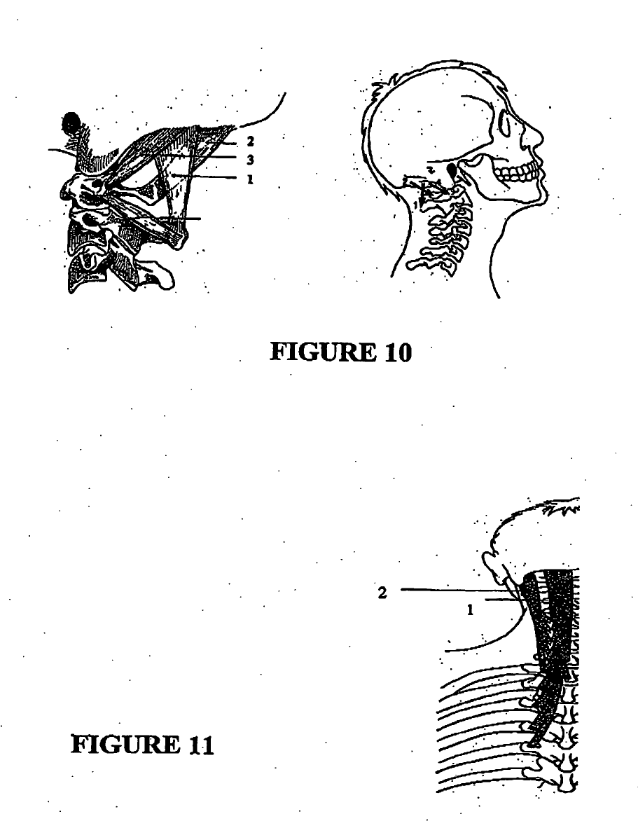

C-C extension is produced by a group of muscles called the C-C extensor muscles. FIGS. 10 and 11 depict some examples of C-C extensor muscles. The primary muscles producing C-C extension are the: semispinalis capitis, rectus capitis posterior major, rectus capitis posterior minor, obliquus capitis superior, splenius capitis, longissimus capitis, sternocleidomastoid, upper trapezius. These muscles all originate from the cervical spine except the upper trapezius and sternocleidomastoid muscles which originate from the clavicle and sternum. The C-C extensor muscles attach to the head (skull) at various distances posterior to the C0/1 articulation and its AOR. The distance the individual muscles are attached from the AOR will determine their lever arm by which they can exert torque at the AOR. Contraction of the C-C extensor muscles therefore exert a force on their bony head attachment, which results in a torque about the AOR via the lever arm which results in the rotation of the head in the sagittal plane.

Muscles that may assist in C-C extension but can not directly contribute to the motion as they do not attach to the head include: semispinalis cervicis, splenius cervicis, cervical multifidus, cervical erector spinae. These muscles assist via their synergistic effects on the cervical spine in support of the C-C extensor muscles.

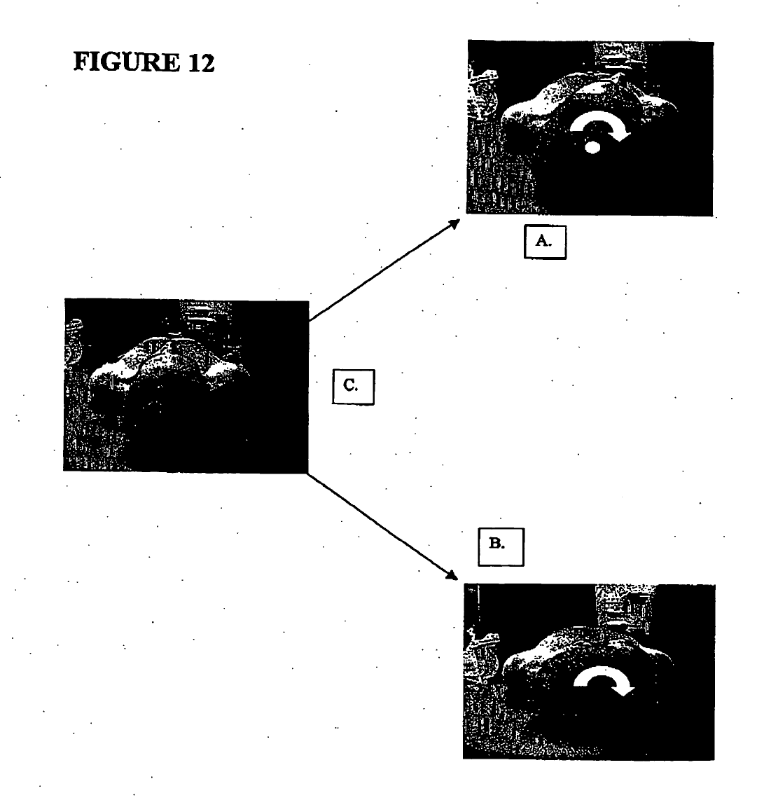

C-C axial rotation describes rotational motion of the head on the neck in the traverse plane such as when a person turns their head to look over their shoulder. Therefore C-C axial rotation can occur to the left or the right side. This rotational motion of the head should occur primarily at the atlanto-axial (C1/2) articulation about the C1/2 AOR as shown in FIG. 12. For practical purposes this axis may be extended to the vertex of the skull (the most superior point of the skull) for alignment to the dynamometer axis. Theoretically this AOR landmark should not translate laterally, and instead should be a rotation point. Axial rotation may be limited to 40 degrees either side to target those muscles producing axial rotation of the C1/2 articulation. Despite the primary articulation of C-C axial rotation being C1/2 motion there will be some motion into axial rotation of the other cervical motion segments due to the associated effects of muscle contraction.

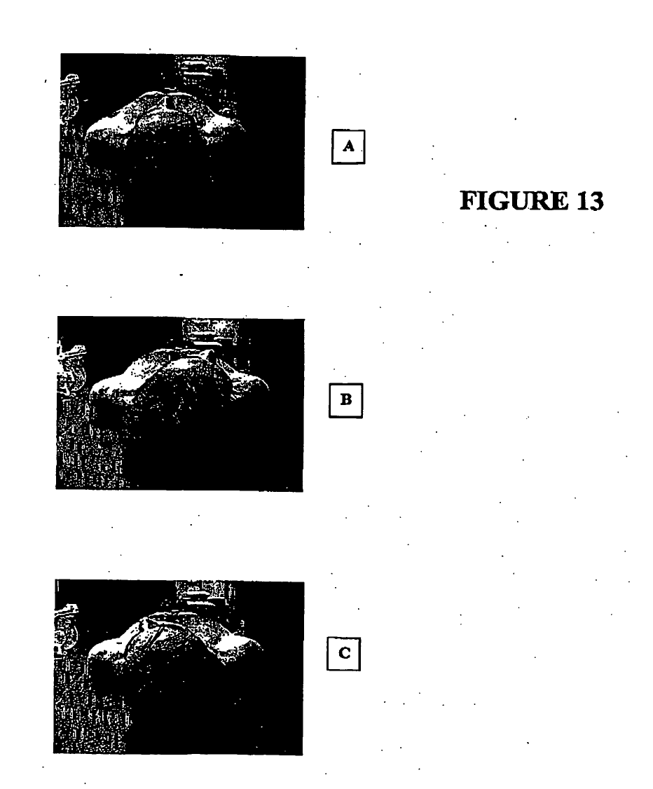

It should be noted that the term C-C axial rotation can be used to describe both a static position of the C-C region and/or a direction of C-C motion. When the C-C region is described as being in C-C axial rotation (or rotated), this means they can only be in a position between the neutral C-C position and the end of range of C-C axial rotation (left or right) as seen in FIG. 13. C-C axial rotation when being described as a direction of C-C motion may occur in any range of transverse plane C-C motion. For example, right C-C axial rotation when performed from the neutral to the end of right C-C axial rotation range is ‘inner range’ right C-C axial rotation. C-C axial rotation may also be initiated from a position at end range left C-C rotation, the motion occurring from end range left C-C axial rotation to neutral is called ‘outer range’ right C-C axial rotation. C-C axial rotation that is initiated at end range left C-C axial rotation and is finished at end range right C-C axial rotation is called ‘full range’ right C-C axial rotation C-C axial rotation motion to the left or right may be initiated or terminated at any point of the transverse plane C-C range.

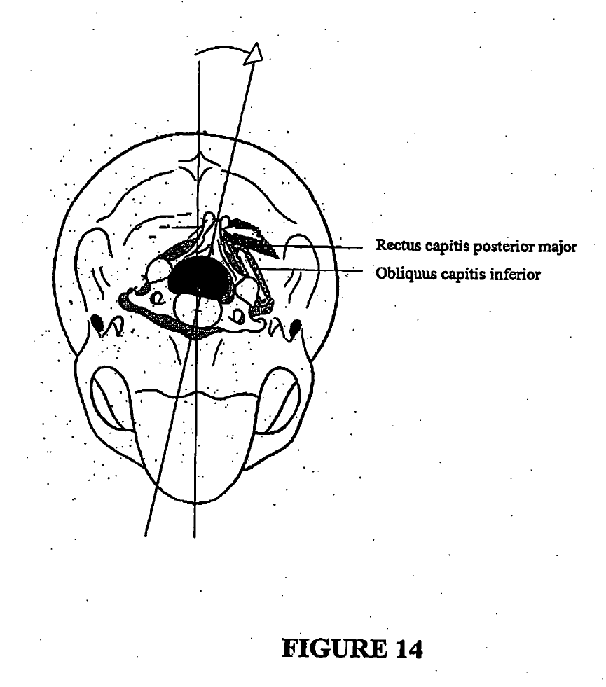

C-C axial rotation is produced by a group of muscles called the C-C axial rotator muscles. FIG. 14 depicts examples of C-C rotator muscles specific to the C1/2 articulation but other muscles that attach to the skull also contribute to C-C axial rotation. The primary muscles producing C-C axial rotation when contracting on one side are the: obliquus capitis inferior, obliquus capitis superior, rectus capitis posterior major and minor, longissimus capitis, splenius capitis, longus capitis, sternocleidomastoid, semispinalis capitis. These muscles all originate from the cervical spine except the sternocleidomastoid muscles which originate from the sternum and clavicle. The C-C rotator muscles attach to the head at various distances lateral to the C1/2 articulation and its AOR. The distance the individual muscles are attached from the AOR will determine their lever arm by which they can exert torque at the AOR. Contraction of the C-C rotator muscles therefore exerts a force on their bony head attachment, which results in a torque about the AOR via the lever arm which results in the rotation of them head in the transverse plane.

Muscles which may assist in C-C axial rotation but can not directly contribute to the motion as they do not attach to the head include: longus cervicis, splenius cervicis, scalene muscles, semispinalis cervicis, cervical erector spine, cervical multifidus. These muscles assist via their synergistic effects on the cervical spine in support of the C-C axial rotator muscles.

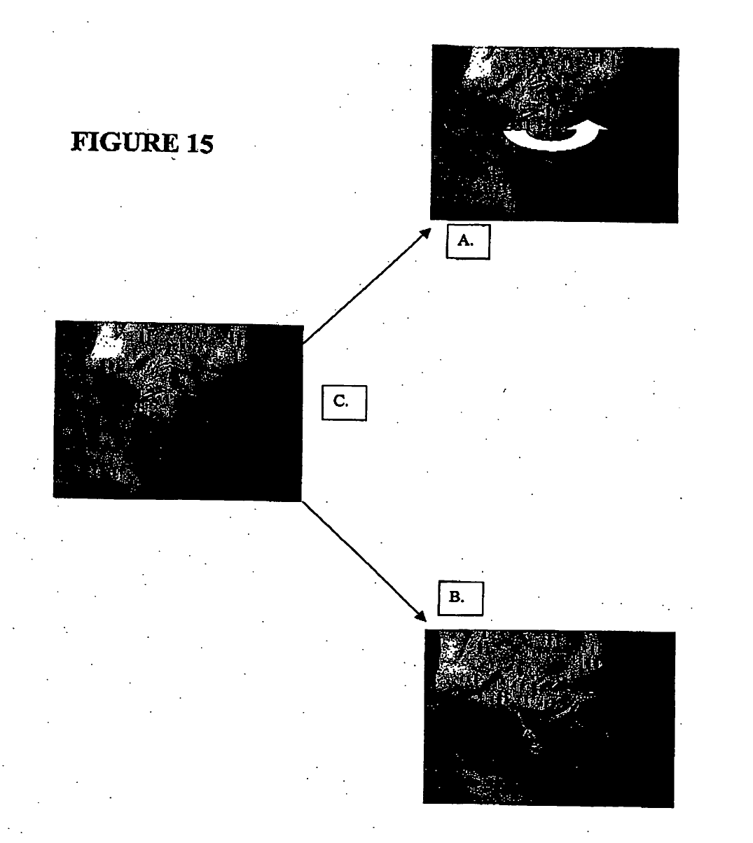

C-C lateral flexion describes rotational motion of the head on the neck in the coronal plane such that in right C-C lateral flexion the right ear approximates the acromion of the shoulder girdle as in FIG. 15A. This rotational motion of the head occurs primarily at the atlanto-occipital (C0/1) articulation about the AOR for C0/1 lateral flexion. Theoretically this AOR landmark should not translate laterally and instead should be a rotation point. This AOR differs from that of lateral flexion of the head and neck which occurs about an AOR around the articulation of the seventh cervical (C7) and first thoracic spine vertebrae (T1) and is associated with a large excursion of lateral translation of the head laterally in the sagittal plant as in FIG. 15B.

Despite the primary articulation of C-C lateral flexion being C0/1 motion there will be some motion into lateral flexion of the other cervical motion segments due to the associated effects of muscle contraction.

It should be noted that the term C-C lateral flexion can be used to describe both a static position of the C-C region and/or a direction of C-C motion. When the C-C region is described as being in right C-C lateral flexion (or laterally flexed to the right), this means they can only be in a position between the neutral C-C position and the end of range of right C-C flexion as displayed in FIG. 15A. C-C lateral flexion when being described as a direction of C-C motion may occur in any range of coronal plane C-C motion. This motion when performed from the neutral to the end of right C-C lateral flexion range is ‘inner range’ right C-C lateral flexion. Right C-C lateral flexion may also be initiated from a position at end range left C-C lateral flexion, the motion occurring from end range left C-C lateral flexion to neutral, and is called ‘outer range’ C-C lateral flexion. Right C-C lateral flexion that is initiated at end range left C-C lateral flexion and is finished at end range right C-C lateral flexion is called ‘full range’ right C-C lateral flexion. Right or left C-C lateral flexion motion may be initiated or terminated at any point of the coronal plane C-C range.

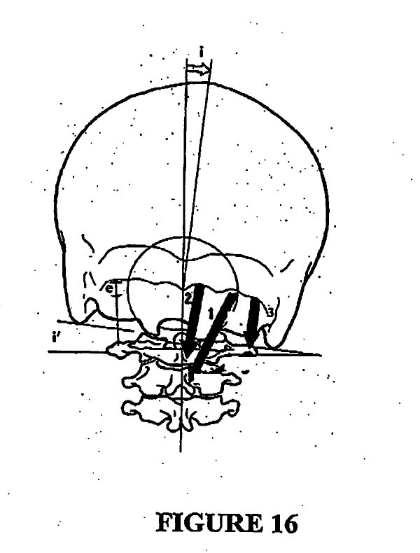

C-C lateral flexion is produced by a group of muscles called the C-C lateral flexor muscles which can be seen in FIG. 16. The primary muscles producing C-C lateral flexion when contracting on one side are the: sternocleidomastoid, obliquus capitis superior, rectus capitis lateralis, longissimus capitis, splenius capitis, and upper trapezius. These muscles all originate from the cervical spine except the sternocleidomastoid and upper trapezius muscles which originate from the sternum and clavicle. The C-C lateral flexor muscles attach to the head at various distances lateral to the C0/1 articulation and its lateral flexion AOR. The distance the individual muscles are attached from the AOR will determine their lever arm by which they can exert torque at the AOR. Contraction of the C-C lateral flexor muscles therefore exerts a force on their bony bead attachment, which results in a torque about the AOR via the lever arm which results in the rotation of the head in the coronal plane.

Muscles which may assist in C-C lateral flexion but can not directly contribute to the motion as they do not attach to the head include: longus cervicis, scalenes. These muscles assist via their synergistic effects on the cervical spine in support of the C-C lateral flexor muscles.

Dynamometry is the use of an instrument for measuring muscular force, or muscular torque. Dynamometry can therefore infer certain aspects of muscle performance by quantifying the torque producing capacity of muscles when actively contracting. C-C dynamometry of the present invention quantifies C-C muscle performance by measuring the torque these muscles impart on the head (skull and jaw combined) when they are actively contracting. C-C muscles produce rotational motions of the head on the articulations between the upper cervical spine and skull and about an anus of rotation (AOR) located in the neck.

Torque occurs about an AOR located in the neck. Torque is calculated by measuring the force the head can exert (via contraction of muscles) to a mechanical lever arm extended at a known distance from the AOR. The force measured in Newtons, is then multiplied by the distance of the lever arm (meters) to give the resultant torque (Newton-meters) about the axis of rotation.

C-C muscle torque may be measured statically or dynamically. In static C-C torque no external motion is permitted. The lever arm is fixed, the muscle contraction is resisted so that no external motion of the head is permitted (isometric muscle contraction). Static muscle torque may be measured at any point in an individual's range of C-C motion. In dynamic C-C muscle torque the lever arm moves with the head measuring torque through the range of motion (isotonic muscle contraction). In this case torque may be measured by the lever arm resisting the motion through range, usually by setting the lever arm to only move at one constant speed. The individual may be asked to move the lever arm as fast through range as possible, by allowing the lever arm to move at one speed the lever arm resists attempts to move it faster and the resultant torque through range can be measured (isokinetic muscle test). This form of measurement can be used when the muscle is shortening (concentric muscle contraction) or when it is lengthening (eccentric muscle contraction).

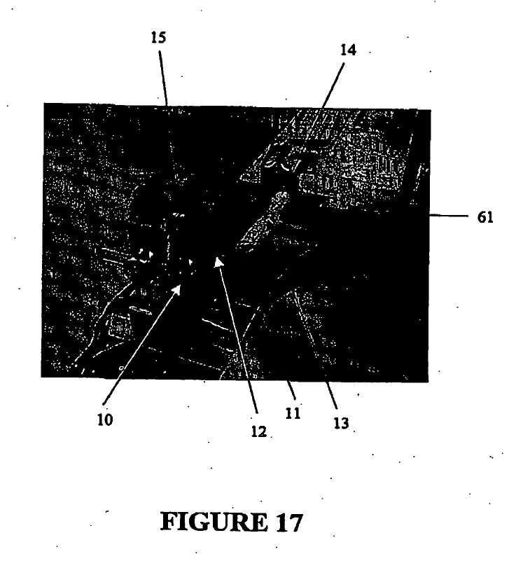

FIG. 17 shows one embodiment of an assessment device for investigating muscular function. In this case and by way of example, the assessment device is a dynamometer 10 which can be considered as an apparatus or device for measuring torque, force, power or work. A lever arm 11 is formed by an extendible section 12 and offset section 13. Preferably the offset section 13 is perpendicular to the extendible section and may have padding 14 for comfortable positioning and operation on a subject 15.

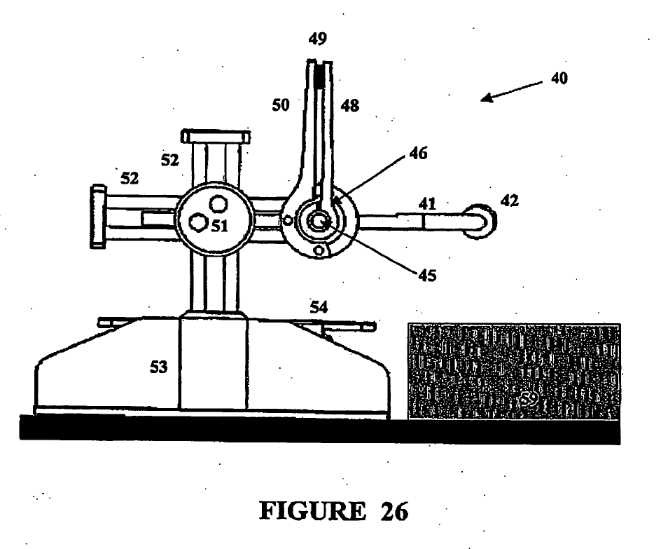

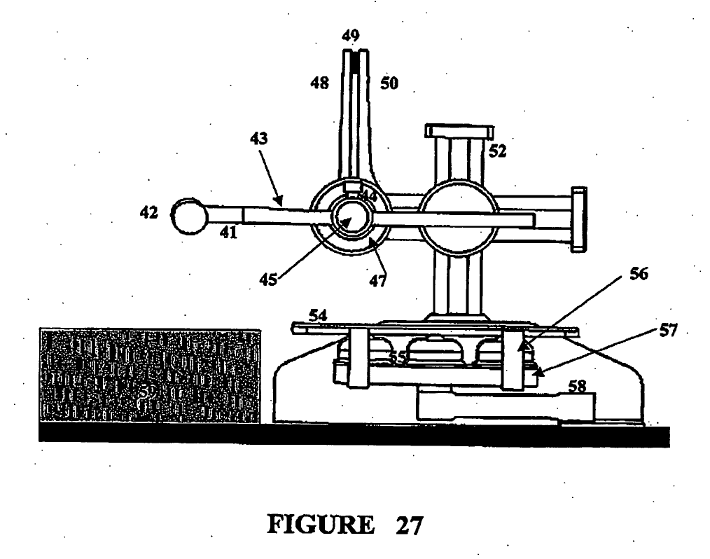

The dynamometer 10 will be further discussed with referee to FIGS. 26 and 27 below. In one embodiment of the method of the present invention, the axis of the dynamometer is aligned with the patients AOR. However, it is possible for other orientations to be utilised provided any repeated or comparative testing is performed with the came arrangement.

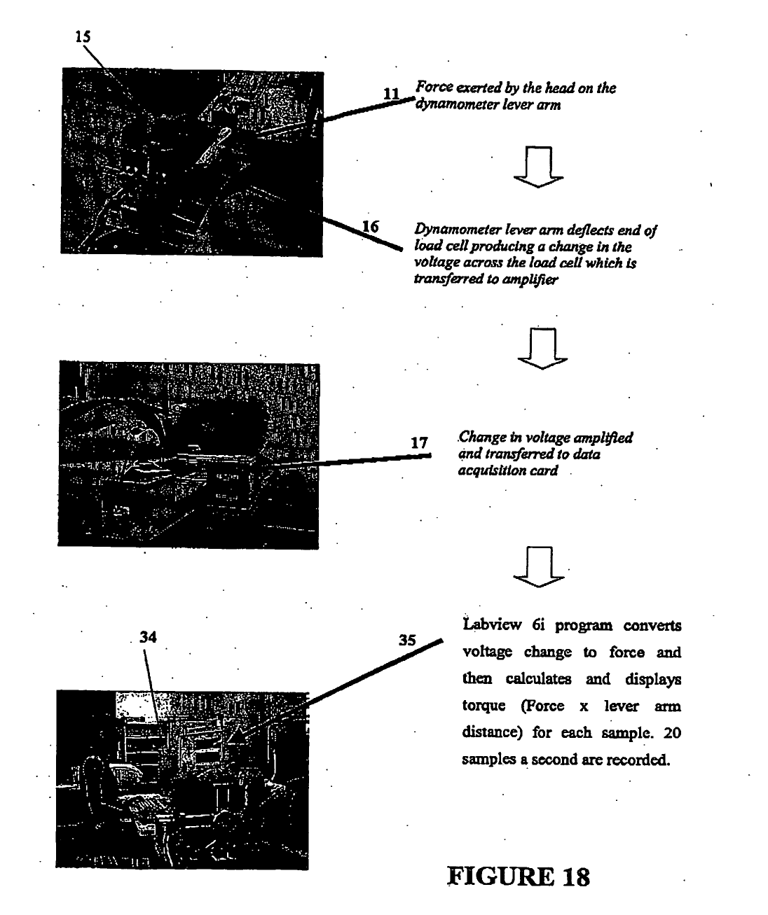

FIG. 18 shows a testing procedure in operation. The subject 15 exerts force onto the lever arm 11 according to instructions from a supervising therapist or technician. The lever arm 11 deflects an end of the load cell 16 to produce a voltage change which is transferred to an amplifier 17. Amplified voltage data is transferred to a computer 34 which is programmed to convert the voltage charge to a force reading and then calculate torque (force×lever arm distance). The lever arm in this calculation may, in an alternative, be an indicator arm to which the force determining means is attached. The computer may also be calibrated to directly convert a amplified voltage directly to the appropriate torque recording. The lever arm distance is preferably variable in operation. Its length may be input manually or automatically determined by suitable electronic arrangement.

C-C muscle torque can be measured and exercise performed statically (no external motion permitted-isometric muscle contraction) in different parts of the persons range of C-C motion or dynamically though range (isotonic muscle contraction both concentrically/shortening or eccentrically/lengthening) via isokinetic (constant speed through range) muscle tests. Muscle performance can be analysed from the torque recordings of the device both statically and dynamically Analysis of muscle performance issues such as strength and endurance can be made, for example:

Strenth—An individuals maximal torque producing capabilities can be assessed by asking the person to perform the contraction ‘as hard as they can’. Peak torque can be measured and this is considered the individuals maximal voluntary contraction (MVC) and is considered by some a measure of strength.

Endurance—Endurance/fatigability can also be tested statically be asking an individual to maintain a contraction at a designated torque intensity for a sustained period of time. This can also be termed a sustained voluntary contraction (SVC). Often this torque intensity will correspond to a percentage of their MVC (eg. 20%, 50%, or 100%) of their MVC. Any target value or range between 5% and 100% may be suitable in appropriate circumstances. Endurance of the muscles is analysed monitoring the deterioration of the torque produced as the contraction is maintained. Dynamically, endurance can also be tested by testing how many repetitions a individual can perform a certain exercise at a specified torque or in a specified time period.

Other performance indicators—Dynamically other aspects of muscle performance such as muscle power and muscle work can also be measured.

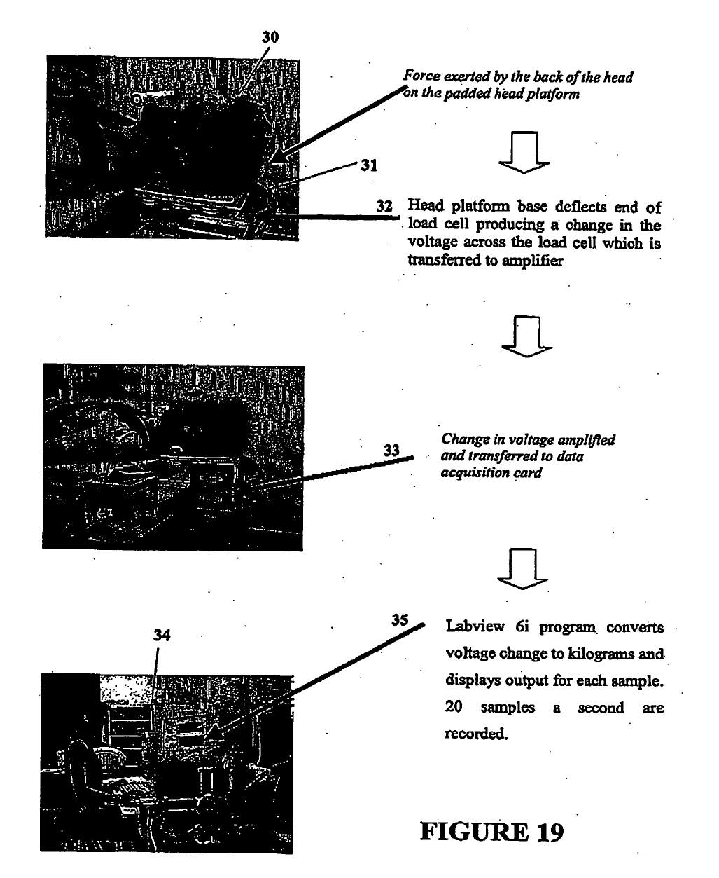

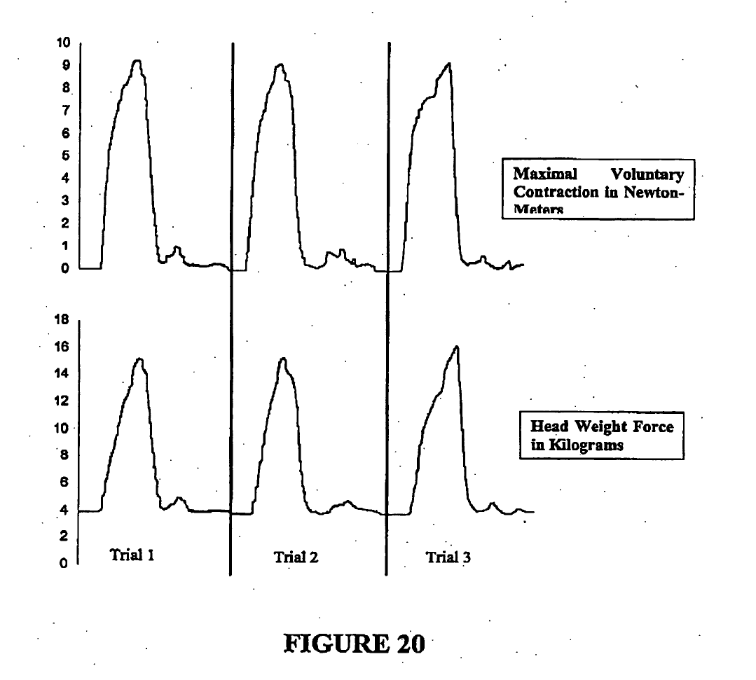

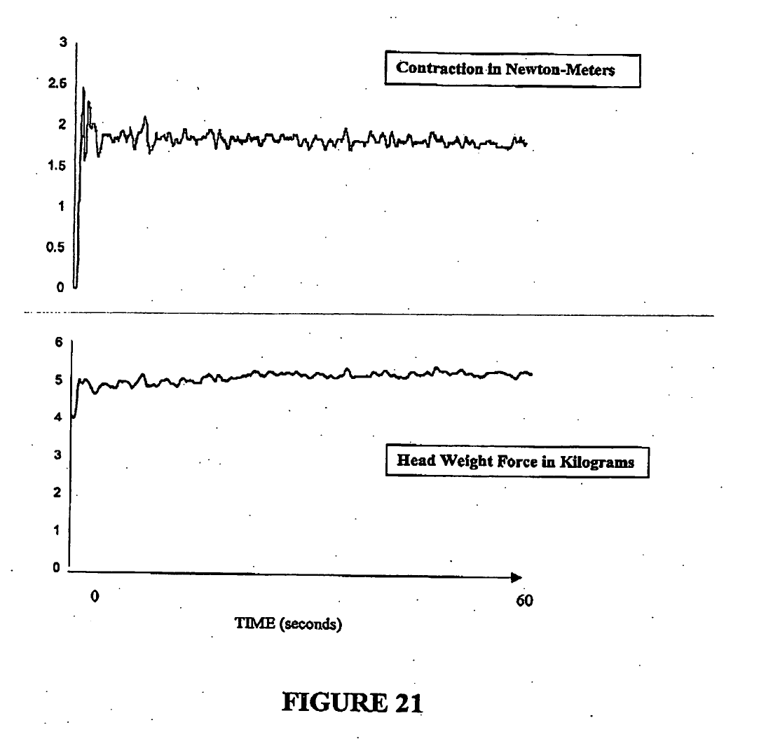

During Cranio-Cervical Dynamometry, the head may remain in contact with the surface it rests on. When performing a muscle contraction, activity of other muscles of the neck may tend to either flex or extend the head and neck on the thorax. The magnitude by which this whole head and neck movement occurs will depend on how well they are performing the C-C torque task. Poor C-C performance may be reflected by a significant change in the tendency for the head and neck to move as a unit. C-C dynamometry may take this into consideration by measuring changes in the force the weight of the head imposes on the surface it rests on. On referring to FIG. 19, it can be seen that the head 30 rests on a platform 31 which is attached to a force transducer 32 which displays the resting head weight and subsequent changes in head weight during the testing procedure. Signals from the force transducer are received at monitoring device 33 and provided to processing means which may be a computer 34 for subsequent analysis and display 35. Representative displays are shown in FIGS. 20 and 21. The method also has the capacity to provide auditory or visual feedback to the participant being tested when a pre-set change in head weight threshold is reached eg. Auditory feedback given when head weight increases or decreases by 10% of the resting head weight.

A clinical trial by the inventors has demonstrated that specific exercise of the muscles that control the head on the neck, the cranio-cervical (C-C) muscles, is an effective strategy to reduce painful neck related symptoms. The present apparatus and method are capable of directly quantifying the performance of the C-C muscles in one or more of the directions of C-C flexion, extension, axial rotation, and lateral flexion. By quantifying an individual's C-C muscle torque capabilities, a rehabilitation program can be tailored for the individual patient based on their unique muscle capabilities and deficits. Consequently rehabilitation programs will be progressed according to the individual's improvement in performance over time, thus for the first time providing a valid means of monitoring and permitting more efficient rehabilitation of the muscles. These exercises can then be performed at the appropriate intensity using the dynamometer and progressed in accordance with the individual's improvement in performance. It also has the advantage of rehabilitating muscles statically and dynamically and addresses all aspects of muscle performance such as strength, endurance, power, work.

This technology has considerable advantages when rehabilitation is in partnership with health insurers who require accurate quantitative measures of muscle rehabilitation status when assessing injury related claims. Both a clinical measurement version of the C-C dynamometer and portable take home version of the C-C dynamometer can be used for rehabilitation purposes. The take home version may be designed for use in the home by patients and may be cheaper, smaller and more portable than the clinic device.

C-C motion involves a rotating action of the head on the neck in a single anatomical plane. In C-C flexion and extension the head rotates on the neck in the sagittal plane, during C-C axial rotation to the left and right the head rotates on the neck in the transverse plane, and during left and right C-C lateral flexion in the coronal plane. When performing C-C motions the head can be considered to be a rigid body with all points on that rigid body moving parallel to the one plane of motion. When a rigid body rotates in one plane there is a point in that body that does not move, has zero velocity, and represents the centre of rotation (COR). An axis through this COR perpendicular to the plane of motion is the axis of rotation (AOR) of the subject. It is through this AOR that torque may be measured although indirect indicators of torque may be advantageously obtained at positions other than the AOR.

If the arc of motion that the rigid body follows is uniform then the axis of rotation will be the same for the whole motion. If the arc is not uniform with different sections of the movement arc involving varying combinations of rotation and translation then each section of the arc will have a different axis of rotation for that motion at that instant. This is termed the instantaneous axis of rotation (IAR) [26]. Due to the non-symmetry of human articulations, the interplay of ligamentous structures and the various lines of muscle actions, motion involving the vertebrae does not result in a uniform arc [27], and this is the case with C-C motion. During C-C motion as described in the testing procedure the location of the AOR is fiber confounded by:
During this test the back of the head rolls on but remains in contact with any surface it rests on. Due to the curved (crest) shape of the back of the head, during through range C-C motion the position of the head in space and in relationship to the axis of the dynamometer changes. The AOR of C-C motion is therefore strongly influenced by the shape of the back of the head when C-C motion is performed with the head resting on the surface such as it is in one described embodiment of the device.
Although C-C motion is primarily of the upper C-C articulations there will also be changes in the curvature of the cervical lordosis in response to C-C motion especially as the head remains in contact with the surface it rests on. During flexion there will be a flattening of the cervical lordosis (cervical flexion), during extension an increase in the cervical lordosis (cervical extension), or during axial rotation some carry over of axial rotation into the mid and lower cervical spine segments. This change in the shape of the mid and lower cervical spine will have effects on the AOR.

The inventors have found two practical, non-limiting approaches to this situation.

### EXAMPLE 1

The first approach is to locate an AOR in the head for the net rotational motion of the head during C-C motion as it is currently performed in the test. This serves to calculate a representative AOR of the head including the effects of the shape of the back of the head and the change in the lordosis as well as the motion of the C-C articulations.

A graphical method was utilised to determine the AOR of a rigid body rotating in one plane (in this case the head) [28]. This method utilises the perpendicular bisectors of the displacement vectors A-A1, and B-B1 of any two points A and B on the rigid body. The intersection of the perpendicular bisectors is the AOR [27]. The technique involves two (or more) reference points A and B. These location of these two reference markers are recorded before motion is commenced (A and B), and again at the completion of the motion (A1 and B1) of the rigid body. If lines (displacement vectors) are drawn from A to A1, and B to B1, and perpendicular bisectors of these two lines are drawn, the intersection of these perpendicular bisectors is the resultant AOR for that motion.

A method was devised using this graphical method in real time using a web camera, computer interface and specialised software. In this way the patients AOR could be located in real time on an x,y coordinate system so that the axis of the dynamometer could immediately be aligned to the individuals calculated AOR. To accommodate measurement of torque at the different points in the range the axis of rotation is then located for specified parts of the C-C range of motion. This method provides a representative axis of rotation for designated ranges of C-C motion eg. whole, inner, outer. Representative AOR could then be located using this method for each specified range allowing the torque to be measured at the AOR specific for that part of the range of C-C motion. Furthermore the computer software program allowed the marking of the location of anatomical landmarks so that the patients head could be positioned with accuracy on separate testing sessions. These landmarks included the tip of the nostril, the tragus of the ear and the chin. Other markers were used to ensure the camera was positioned consistently in relationship to the dynamometer.

Preliminary studies demonstrated concurrent validity of this method by showing there was no significant difference between the calculated AOR of a rigid body rotating in one plane and a known AOR (trundle wheel) (p>0.42). This method was shown to have good reliability to locate the AOR (x,y coordinates in millimeters (mm)) in 20 participants when the AOR was calculated on two separate days (SEM x=3.88 mm, y=8.98 mm, ICC x=0.938, y=0.738).

The procedure is described as follows:

Patient Position—The participant was placed in the supine position with the legs elevated and suspended on slings such that the knees and the hips were flexed to 90 degrees. This position was used as it suspended the legs and the participant was less likely to be able to pull down with the legs on a solid object therefore corrupting later torque measurement. The participants head was placed in neutral according to the Frankfort Plane (FIG. 2). The participant was also to fold the arms as would be the position for later torque measurement.

Reference Markers—Adhesive markers were then placed over the anatomical reference landmarkers A and B and a black dot was placed on the adhesive marker to indicate the exact point for the digitisation of the reference marker later in the procedure. The specialised software was then used to locate the starting neutral points (chin, tip of the nostril, notch above the tragus of the ear). The recording of these three points allowed the head to be positioned in the same location with respect to the dynamometer on subsequent testing sessions. Three camera reference digitised points were also located so that on later testing sessions it could be checked that the camera was in the same position in relationship to the dynamometer and therefore the participants head would be in the same position in relationship to the dynamometer.

Full Range C-C flexion—The participant was then shown the C-C extension-flexion motion and was asked to practice the motion until familiar with the movement required. They were then asked to roll the head back to their familiar comfortable range of C-C extension, the location of the tip of the nostril was then marked with “E” marker to represent end range extension. The participant was asked to C-C flex until they reached their comfortable active end of range. The location of the tip of the nostril was then marked with the “F” marker to represent end range flexion. Once the full range is marked the AOR location can then be performed. The participant is asked to move to full C-C extension, at this point the reference markers A and B are recorded, the participant is asked to move to full C-C flexion, the investigator ensuring that the tip of the nostril reaches the “E-F” markers. At the full C-C flexion range the participant is asked to maintain the position while the location of the reference markers A1 and B1 are recorded. The software is then programmed to draw displacement vectors between the points A-A1, and B-B1. Perpendicular bisectors off these displacement vectors are then drawn, the intersection of these perpendicular bisectors is the AOR for that trial and is also recorded as x,y coordinates. 5 trials are performed and an average AOR is calculated representing the average x and y coordinates. The software was then able to calculate the angle of C-C motion performed in this manner, this angle was then divided into an inner (50-100%), an outer (0-50%) and a middle (25-75%). These ranges represent motion around the mid point of the inner range (75%), outer range (25%), and middle range (50%) for later torque measurement.

Inner AOR calculations—A line was drawn representing the 50% mark of the full range, the participants head moved until the tip of the nostril reached this line. The “E” marker was then placed to mark the starting point of inner range C-C flexion. The same procedure to locate the average AOR location as for full range was then repeated for inner range motion, that being C-C flexion from the new “E” position to the “F” position.

Outer and middle AOR calculations—The same procedure as for inner was performed for the middle and outer ranges. For outer range the “F” marker was placed at the 50% mark to represent end of range outer C-C flexion. For the middle range the “E” and “F” markers were placed at the 25% and 75% points respectively. 5 trials were performed using the same procedure as described full range to find the average AOR for each range.

Advantages of this method:
Gives a representative AOR of the head for the C-C test as it is performed currently with the head resting on a flat platform in the supine (lying on back). ie. it takes into consideration not only C-C articular motion and C-C mule influences, but also the effects of the shape of the back of the head and the change in the cervical lordosis brought about by motion of the middle and lower cervical spine.

Disadvantages of this method:
Primary AOR corruption—Although AOR calculated in this method may be representative of the AOR of head rotation during the test, it can be argued that it is not representative of C-C motion as there are too many corrupting factors especially the shape of the back of the head and the motion of the mid and lower cervical spine.
Multiple procedural steps—The procedure involves many procedural step, each step increasing the chance of inaccuracy.
Parallax error—The procedure involves the use of a web camera to align landmarks that are of different distances from the camera, there will be some inaccuracy due to parallax error.
Patient performance of Non-planar motion—It was found with this technique that slight deviation by the participant away from pure sagittal motion produced significant deviation of the located AOR as opposed to the location if the participant performed pure planar motion. The technique therefore required the participant to be skilled in the performance of the C-C action. This may be challenging especially when performed on individuals with painful dysfunction of their neck with the inevitable alterations in articular motion, muscle control, and kinaesthetic awareness (body awareness).
Patient performance of position holds—This technique also required the participant to maintain still positions when the reference markers (A and B) were being digitally recorded. This was especially so between the time it took to record the location of the first and second marker (ie. the time between marking location A on the nose and location B above the eye). This was especially difficult to achieve when the position to be maintained involved a challenging muscle position to be maintained as in the case on inner range C-C flexion. It was noted that despite best efforts of patient and investigator to maintain the still position the patient often did move slightly before both points could be marked. This may be a significant source of error and accounts for the large standard error of the measurement for the inner rang of the test.
Moving skin markers—The position of the references markers A (osteo-chondral junction of the nose) and B (zygomatic process of the frontal bone just above the eye) were chosen partly as they were thought to be areas of the skin not greatly affected by movement of the skin over the bony landmarks during motion of the head. Sites on the mandible were not used due to the confounding effect of motion of the mandible on the cranium. It was noted however during the procedure that eye related motion would change the position of the landmark therefore inducing error. The same was for movement of the nose although this appeared less of a problem.

### EXAMPLE 2

This method was directed to AOR specific to primary articulations. This method used known bony anatomical landmarks that are representative of the AOR for motion of the articulations primarily targeted during C-C motion. For example the primary C-C articulation of C-C flexion and extension is the C0/1 (atlanto-occipital) motion segment. The radiographic located AOR for C0/1 flexion/extension occurs about a point slightly superior and anterior to the mastoid process of the skull (FIG. 5,8) [29-31] perpendicular to the sagittal plane. Torque measured about this AOR would represent the torque of the CC flexor or extensor muscles about the primary AOR of C-C flexion and extension. For C-C axial rotation the axis of rotation is near the vertex of the head perpendicular to the transverse plane and in line with the axis of rotation of the C1/2 (atlanto-axial articulation) articulation (FIG. 12), the principle articulation for C-C axial rotation. It is acknowledged that these motions do not occur in isolation purely on these articulations and that in biological articulation the axis of rotation is constantly altering through range. However alteration in AOR through range of these single articulations is not large.

For this AOR technique a web camera has also been used to ensure the head and dynamometer are positioned consistently on separate days as for Example 1. However clinically this would not be necessary and the dynamometer could physically be aligned to the patients AOR using the above described AOR landmarks, and ensuring the angle and the length of the lever arm remain constant. This is one of the significant reasons for measuring lever arm length so as to ensure consistency in repeat applications.

For this technique the dynamometer axis was aligned at the superior and anterior part of the patients mastoid process such that it was just posterior to the external acoustic meatus (earhole). This was the landmark chosen as approximating the anterior/superior mastoid process and the C0/1 AOR (FIG. 17). The axis remains the same for the middle, inner, and outer ranges. As the motion is primarily focused to the C0/1 motion segment the range is much smaller then for the first method.

Advantages of this method:
Minimal procedural steps;
Easy to adapt to a clinical application;
Easy to replicate the positioning of the dynamometer axis to the patients AOR;
The lever arm remains constant for all ranges tested, therefore torque can be properly compared between different parts of the range.

Disadvantages of this method:
Does not take into account the effect the shape of the back of the head or the motion of the mid and lower cervical motion segments will have on the AOR.

The method of the second example may be preferred because:
It was decided that if the dynamometer were to primarily test the torque of the muscles that produce C-C motion it should be measured about the AOR of the articulations primarily responsible for C-C motion. Namely the C0/1 motion segment for flexion/extension, the C1/2 motion segment for axial rotation, and the C0/1 motion segment for lateral flexion.
There is research evidence (including radiographic evidence) of the location of the AOR of the C-C articulations [29-31].
Practically the second method is a simpler process to align the axis to a bony landmark on the head, and may be replicated more accurately on sequential testing sessions. This has considerable advantages when used as a clinical tool.
In the first generalised method the C-C dynamometer axis is aligned to an x,y coordinate representative of the location of the AOR located on the individuals head. The problems encountered with this method included:
   The generalised method involves many more procedural steps, inviting inaccuracy, and requires the use of a web camera, and specialised software and a more elaborate setup. Clinically this makes the first method much less attractive when using C-C dynamometry as a clinical tool.
   It was found that the representative AOR located in this manner differed considerably for the 3 portions of the C-C range of motion ie. inner, middle, outer. Therefore when the C-C dynamometer was set up for these axes the dynamometer lever arm was different for each of the 3 parts of the range. Therefore torque was strongly influenced by the dynamometer set up (ie. longer lever arm will produce greater torque for the same force). It was considered that this had the tendency to cause incorrect comparisons of torque produced at the different parts of the C-C range of motion. This made dynamic torque measurement through range very difficult to apply.
Due to the curved (crest) shape of the back of the head, during through range C-C motion the position of the head in space and in relationship to the axis of the dynamometer changes significantly. The AOR of C-C motion is therefore strongly influenced by the shape of the back of the head. It may be of advantage if the influence of the back of the head is eliminated so that the AOR is representative of the C-C articulations. This may be achieved by either suspending the weight of the head and restraining rotational motion to the axis of rotation of the head or by changing the weight bearing contact point from the back of the head to the suboccipital region. Testing may also be performed in sitting position which would eliminate the weight of the head having to be suspended from the axis of rotation. It is preferred to target the AOR of the primary articulations at which the C-C motion occurs as previously found through scientific methods.

It is preferable if the axis of the dynamometer is physically aligned at the landmark of the patients AOR. Firstly the patient may be positioned so that they are aligned to the dynamometer axis in the coronal plane, the dynamometer axis will then be adjusted in the transverse plane to align to the patients AOR. The lever arm is extended and fixed at a length so that it fits under the chin for flexion, at the front of the chin for extension, and on the side of the head for rotation and lateral flexion. Once the lever arm is set up for the patient it can be replicated in length and angle with the use of a potentiometer. Therefore the positioning of the dynamometer axis, and lever arm can be replicated easily over separate sessions.

While the dynamometer may be used in the supine position, it may also be used in the sitting position which may replicate functional muscle performance more accurately.

The measurement of torque is generated through the use of a load cell transducer, connected to an amplifier, a data acquisition card, and a Laview virtual instruments program. The patient exerts force through the C-C dynamometer lever arm 11 at a known distance form the AOR and this force is transferred to one end of the load cell. The other end of the load cell 16 is stationary. This force at one end of the load cell causes a bending deflection of the load cell which produces a change in the voltage across the bridge of the load cell. The larger the deflection, the larger the voltage change. This change in voltage is transmitted to an amplifier and data acquisition card 17 and to a computer software program (Labview program) where the product of the force measurement (newtons) and the lever arm distance (meters) to give a displayed output of torque (Newton-meters). The data is sampled at 20 Hz. Therefore torque can be measured over specified time periods and specified C-C range of motion. See FIGS. 20 and 21 for examples of torque output recordings for maximal voluntary contraction and endurance tasks. This process has been discussed above.

Other forms of torque measurement may be used.

Torque sensors and torque instruments may be used to measure torque in a variety of applications. Torque sensors are categorised into two main type, reaction and rotary. Reaction torque sensors measure static and dynamic torque with a stationary or non-rotating transducer. Rotary torque sensors use rotary transducers to measure torque.

The technology of torque sensors can be magnetoelastic, piezoelectric, and strain gauge. A magnetoelastic torque sensor detects changes in permeability by measuring changes in its own magnetic field. A piezoelectric material is compressed and generates a charge, which is measured by a charge amplifier. To measure torque, strain gauge elements may be mounted in pairs on a shaft, one gauge measuring the increase in length (in the direction in which the surface is under tension), the other measuring the decrease in length in the other direction.

Torque sensors can be provided as many different types of devices including sensor element or chip, sensor or transducer, instrument or meter, gauge or indicator, and recorder and totalisers. A sensor element or chip denotes a “raw” device such as a strain gauge, or one with no integral signal conditioning or packaging. A sensor or transducer is a more complex device with packaging and/or signal conditioning that is powered and provides an output such a dc voltage, a 4-20 mA current loop, or similar. An instrument of meter is a self-contained unit that provides an output such as a display locally at or near the device, typically, also including signal processing and/or conditioning. A gauge or indicator is a device that has a (usually analog) display and no electronic output such as a tension gauge. A recorder or totaliser is an instrument that records, totalises, or tracks force measurement over time and includes simple data logging capability or advanced features such as mathematical functions and graphing.

Common outputs for torque sensors include analog voltage, analog current, analog or modulated frequency, switch or alarm, serial, and parallel. At least some of these may be converted to digital signals.

In C-C dynamometry the force exerted by the head on the surface 31 it rests on may be informative regarding strategies the patient will use to achieve C-C torque. Before C-C torque is commenced the mass of the head is measured at rest. This is achieved by a load cell 32 under the head platform. The load cell is secured and suspended above the bench the patient is lying on, and at the other end is attached to the platform the head rests on. The weight of the head therefore vertically deflects one end of the load cell, causing a bending moment across the load cell and a change in the voltage across the load cell. This voltage change is amplified, and recorded in a data acquisition card 33 and is displayed 35 and recorded on a labview Virtual Instruments program as weight of the head in kilograms. When C-C torque is commenced any lifting or pushing back of the head is registered by the change in deflection of the load cell and corresponding weight output. FIGS. 20 and 21 show an example of head weight force output during maximal voluntary contraction and endurance tests.

Once the participant AOR and the Dynamometer axis are aligned the dynamometer chin lever arm is positioned so that the padded chin bar sits on the flat inferior border of the mandible for C-C flexion (see FIG. 17). It must be positioned such that it is not too low and therefore physically compressing the larynx, or too high on the chin so that it is in danger of slipping off the end of the chin with skin motion. The patient is asked to hold the lever arm firmly onto the chin so that the lever arm can be tightened at the correct length. The patient is asked to C-C flex against the chin bar to ensure it feels stable and that the chin sits snugly against the padded bar. Once the therapist and the patient are satisfied the bar is in the correct position it is located on the software program as the dynamometer middle position. The patients head is then C-C extended by 10 degrees and the same occurs to position the outer range torque measurement position. It is then moved 10 degrees into C-C flexion from the middle range to position the bar in the inner range torque measurement position.

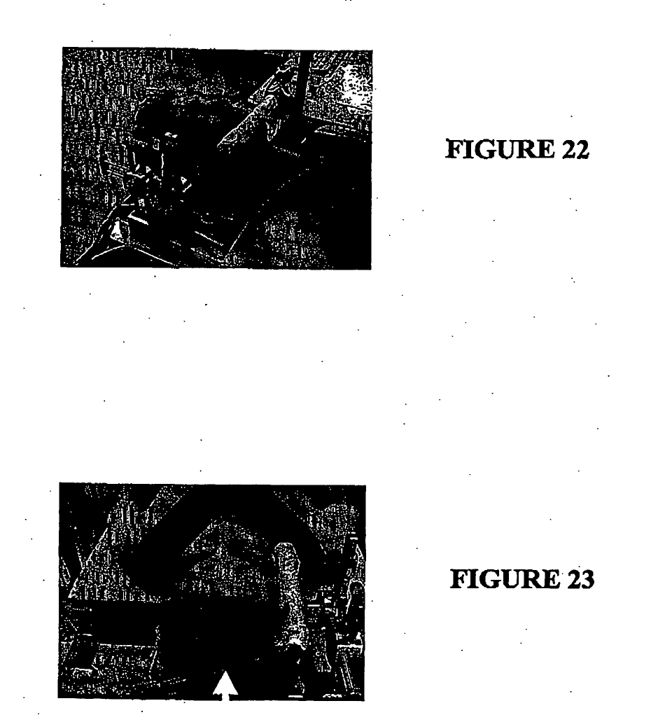

For C-C extension, the AOR set-up is similar to that of C-C flexion and is shown in FIG. 22. The padded chin bar is position on the front of the mandible so that there is no pressure on the lower teeth. Care must be taken to avoid pressure on the teeth. As for flexion torque is measured at different ranges of C-C extension.

For C-C axial rotation, the dynamometer axis is set-up at the vertex of the head (FIG. 12). As shown in FIG. 23, the torque lever arm of the dynamometer is extended from the AOR so that the padded bar is positioned on the side of the head just posterior to the lateral portion of the orbit of the eye and along the maxilla. C-C axial rotation involves approximately 40-45 degrees of rotation of the head on the neck in either direction. Torque for example in the direction of right C-C axial rotation may be measured in the range of 40 degrees of left rotation to 40 degrees of right rotation.

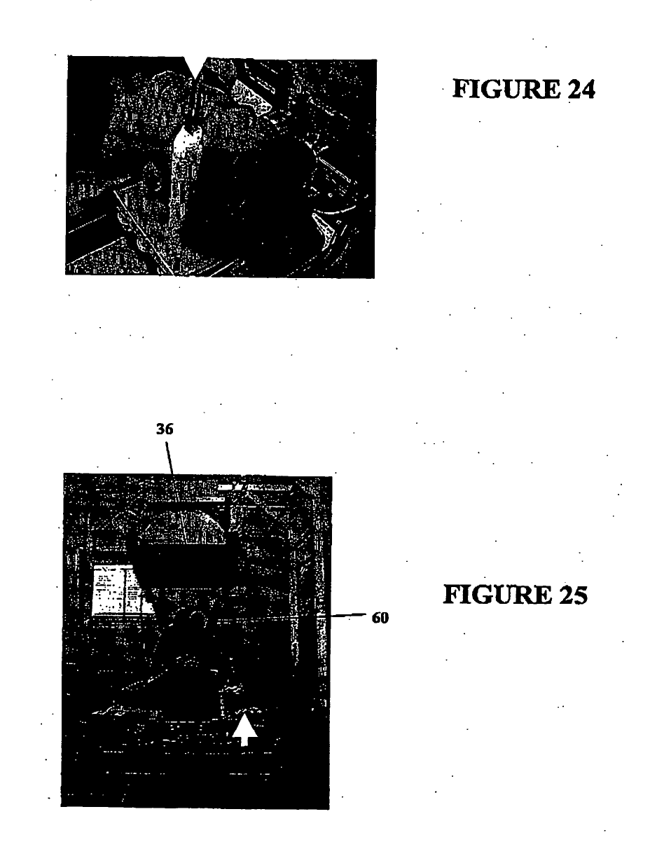

The set-up of FIG. 24 is suitable for C-C lateral flexion. The same landmarks may be used in the sitting position.

When measuring torque, one of the above set-ups is used. The set-up will also be determined by the type of muscle contraction to be performed. If isometric muscle contractions are to be tested the patients head and C-CD lever arm will be positioned in the predetermined position in the C-C range to be tested. If isotonic muscle contractions are to be performed the patients head and lever arm are positioned at the beginning of the C-C range of motion to be tested.

### EXAMPLE 3

### Isometric Maximal Voluntary Contraction Muscle Test

The patient and C-CD are positioned corresponding to the patients AOR and in the predetermined C-C range as described above. The patient is asked to ‘nod their head such that their jaw pushes down onto the padded bar so that the head remains in contact with the surface it rests on’. The patient is asked to perform C-C flexion gently at first to warm up. When the therapist is satisfied the test is performed correctly the patient is asked to repeat the test but this time to perform the action as hard as possible. The patient is given visual feedback (FIG. 25) through an overhead display 36 of the increase in torque with their increasing effort. The visual feedback is displayed as an elevating mark on a graph. A rest period is given between trials. FIG. 20 is an example of the output for this test. A test-retest study has been performed to assess the reliability of C-C dynamometry in the measurement of C-C flexor muscle performance using this procedure over two separate sessions spaced 2 weeks apart (to minimise carry-over effects such as those from training or learning between sessions). Intraclass Correlational Coefficients (ICC) and the Standard Error of the Measurement (SEM) reliability indicies have been calculated. Analysis of data (n=20) has demonstrated good reliability for MVC torque measurement between sessions (ICC 0.9243, SEM 0.17 newton-meters).

### EXAMPLE 4

### Isometric Endurance

A predetermined percentage (eg. 20 or 50%) of the person's maximal voluntary contraction or an arbitrary torque intensity is chosen. A visual display gives the patient the torque intensity level required. The participant is asked to ‘nod their head such that their jaw pushes down onto the padded bar to achieve the pre-determined torque intensity and is asked to maintain this torque intensity as accurately as possible for a predetermined time period or until the torque level decays beyond a certain level (eg. 50% of the pre-determined level). This may also be called a sustained voluntary contraction (SVC). FIG. 21 is an example of the recorded output from this test. Variations of these tests can be performed so that the patient may be asked to control the torque output only, or both torque and head weight force simultaneously.

The mechanical components of a preferred embodiment of the apparatus of the present invention are shown in FIGS. 26 and 27. The dynamometer 40 comprises:

1) Dynamometer Lever Arm 41—This lever arm is adjustable in length from the dynamometer axis so that it fits snugly against the surface of the head where it will resist C-C motion in the designated direction.

2) Head Contact Padding 42—The dynamometer lever arm is padded at the contact site of the head. This may be a simple layer of foam or have a more moulded surface that contours the area of the head it contacts for improved patient comfort and mechanical transmission of forces.

3) Lever arm distance indicator 43—Marks at known distances from the head contact point indicate the distance of the lever arm (axis to the head contact point). This distance may be used in the calculation of torque, however, it is also important for consistency of alignment in repeat tests. A potentiometer may be used for a quicker method of distance calculation

4) Lever arm bolt 44—This bolt is used to secure the lever arm once the appropriate distance of the lever arm has been found.

5) Dynamometer axis 45—This axis may be aligned to the patients designed AOR and is the point about which torque is measured. The axis is able to be mobile for adjustment of the lever arm angle to accommodate all ranges of C-C motion for isometric torque measurement. Once the appropriate length and angle is found the axis is then locked onto the torque arm by tightening the axis bolt so that the lever arm becomes rigid and all force induced by the head on the lever arm is transferred to the dynamometer torque arm which transfers the bending force to the load cell. The axis may also be able to be freely mobile (minimal friction) for the measurement of dynamic torque (isokinetic). Positioning of the axis may be varied for C-C rotation (vortex of head) and later flexion. The latter may require inward extension arrangement to position on the sagittal plane of the subject around an anterior (posterior line through C₀/C₁.

6) Axis Bolt 46—This bolt can be tightened and loosened to allow the dynamometer axis to be rigid or freely mobile.

7) Axis Housing 47—This contains ball bearing which minimises friction of the axis when the axis bolt is loosened so that the axis and therefore the lever arm angle is adjustable.

8) Dynamometer Torque Arm (mobile) 48—This arm is locked to the dynamometer axis so the transference of force can be directly transferred to the load cell.

9) Torque Load Cell (LOAD CELL 1) 49—Please see description below (Electronic components) for specification of one suitable load cell. This load cell is secured at one end by the rigid dynamometer arm and secure at the other end by the dynamometer torque arm. Thus motion of the dynamometer torque arm can induce a bonding force on the load cell, which changes the voltage across the load cell, which is amplified and converted to a force measure and a torque measure. The torque is calculated by multiplying the force by the distance to the torque load cell from the AOR. The torque arm may be considered an indicator arm, at least in respect of the operative distance for calculating torque.

10) Rigid dynamometer torque arm 50—this arm is rigid with the axis housing of the dynamometer and is attached to the force load cell. It provides a rigid base for the load cell so that motion of the dynamometer torque arm will produce a bending moment on the load cell.

11) Axis adjustment unit 51—This casing houses bolts which can be loosened and tightened to allow the adjustment of the dynamometer axis in the horizontal and vertical directions.

12) Axis adjustment rods 52—These rods are used as guides from which the axis position can be adjusted to align to the patients AOR and then locked into position. The rods and As adjustment unit may form a support frame.

13) Dynamometer base plate and block 53—This provides a rigid attachment for the dynamometer to the bench upon which the patient rests.

14) Padded Head platform 54—This is the platform the head rests on which is mobile in the direction of C-C motion required so that torque is not lost due to friction of the back of the head on the surface it rests on.

15) Ball Bearings 55—The padded head platform sits on top of the ball bearings which allow the platform to move with the head so torque is not lost through friction.

16) Platform guides 56—These guide wheels ensure the head platform moves along the plane of motion of the head.

17) Head platform base 57—This structure supports the ball bearing, platform guides and padded head platform and is attached onto the end of the head weight load cell (LOAD CELL 2) such that a change in the force exam onto the padded head platform by the head during procedural tests is detected by this load cell ie. if the head lifts or is pushed back in response to the torque testing.

18) Head force load cell (LOAD CELL 2) 58—This load cell is a single point load cell and responds to bending forces which changes the voltage across the load cell which is converted to a meaningful force output. The other end of the load cell may be attached rigidly to the bench upon which the patient rests or other suitable configuration.

19) Rigid Bench with Padding or plinth 59—This padded bench is at the same horizontal level of the head so that the spine can be positioned in a neutral position for testing (FIG. 25).

20) Leg slings (FIG. 25) 60—the legs of the patient are elevated on slings which are mobile so that the hips and knees are bent to approximately 90 degrees and so that the legs can not purchase onto solid surface as this may allow the patient to exert unwanted additional torque by pulling down with the lower limbs.

21) Restraining straps (FIG. 17) 61—these adjustable straps allow the patients shoulders and to be restrained so that torque exerted on the chin will not be lost by movement of the body.

Suitable electronic components may include:

Load Cell 1—TBS Series, Thin beam sensor. FIGS. 27 #49)
Manufacturer—Transducer Techniques
Supplier—Davidson Measurement PTY LTD
This load cell was used for the measurement of force used in the calculation of torque.

Load Cell 2—ESP Series, Single point load cell. (FIG. 27 #58)
Manufacturer—Transducer Techniques
Supplier—Davidson Measurement PTY LTD
This load cell was used for the measurement of force exerted by the change in head force on the head platform.

Digit Panel Display (×2), PM4-SG-240-5E-A (FIG. 18 b)
Powered: 240 VAC
Input: 0.5 to 10 mV/V selectable
Output: 1× relay and Analogue (4-20 mA, 0-1V and 0-10V selectable)
Supplier and Manufacturer—Davidson Measurement PTY LTD
These displays panels were used to collect and amplify the voltage output from the two load cells.

Software for the conversion of amplified voltage recordings received in the data acquisition card used a Labview 6i Virtual Instruments Program, National Instruments Corporation.

Optional beneficial features of the clinical device may include:
Adjustable axis of dynamometer for measurement of C-C axial rotation and lateral flexion torque.
The measurement of combined movement torque.
An electronic motor may be applied to allow measurement of torque isokinetically as well as isometrically.
Unwanted head motion can be restrained so that motion is permitted only around the AOR ie. Eliminates the effect of the shape of the back of the head on the AOR.
Can be performed sitting or supine.

The use of cranio-cervical dynamometry instruments that measure and rehabilitate neck muscles is disclosed in this specification. The devices feature mechanical principles that permit graded resistance to both upper and lower functional neck muscle groups. Graded resistance permits quantification of neck muscle performance and therefore offers a method of assessment and graded rehabilitation. There arm two preferred devices that have the same principles of use but differ in their sophistication, and portability making one appropriate for precise measurement within a clinic and one appropriate for use for rehabilitative exercise within the home.

### EXAMPLE 5

### Specificity of Neck Muscle Usage with the New Devices

Myoelectric measurements of neck muscle activity were recorded on 10 individuals when performing exercises with the devices of the present invention at maximal, moderate and low contraction intensities as may be expected when performing exercise within a clinic. The aim of the experiment was to establish if the method targeted key neck muscles (indicated by the arrow in Graphs below) known to be problematic in neck pain disorders. The results are shown in the graphs below. For muscle tests at all intensities (low, moderate, high) the key target muscles were shown to be the most active of all the neck muscles validating that the exercise performed with these devices is specific to the key muscles and will best measure and rehabilitate their performance.

[image]

### EXAMPLE 6

### Deficits in Neck Muscle Performance in Individuals with Neck Pain

Comparisons were made between the measurements of neck muscle performance using the devices of the present invention in persons with neck pain (n=46) and persons without neck pain (n=47). Measurements were made regarding strength endurance, and capacity to sustain a steady muscle contraction. All these measures arm considered important in the management of neck pain. As depicted below, the Neck Pain group performed significantly (p<0.05) poorer then the control group on all measures of performance. This is evidence that the devices can be used to measure neck muscle impairment and are therefore a valuable clinical tool.

[image]

Features of the version for home use may include the following:
Able to perform C-C and cervical muscular exercise in the directions of flexion, extension, axial rotation (left and right) exercise, lateral flexion (left and right).
Able to perform exercise statically (isometrically) in any point of the CC range or dynamically (isotonically) through range therefore the dynamometer axis is able to be locked not permitting motion or is able to move through range.
Dynamic exercise is able to be performed against predetermined resistance via a shock absorber or spring/rubberband resistance. This is achieved by the resistance restraining the motion of the dynamometer lever arm that the individual is moving against.
Dynamic (Isotonic) exercise can be performed concentrically (muscle fibres contracting but shorten) or eccentrically (muscle fibres contracting but lengthen) against predetermined resistance.
Dynamic exercise may also be performed against variable resistance through range (isokinetic muscle contraction).
Visual feedback can be given to the patient regarding their level of muscular effort.
The back of the head rets on a surge which mobile and minimises friction to prevent unaccountable resistance from the friction of the back of the head sliding on the surface it rests on.
Portable and able to be used on the floor for use lying down or attached to a vertical surface such as a door to be used in a sitting position.
Adjustable dynamometer axis in the horizontal and vertical directions to be adjustable to individuals axis.
Adjustable dynamometer lever arm to allow adjustment for measurement of C-C flexion, extension, and axial rotation.

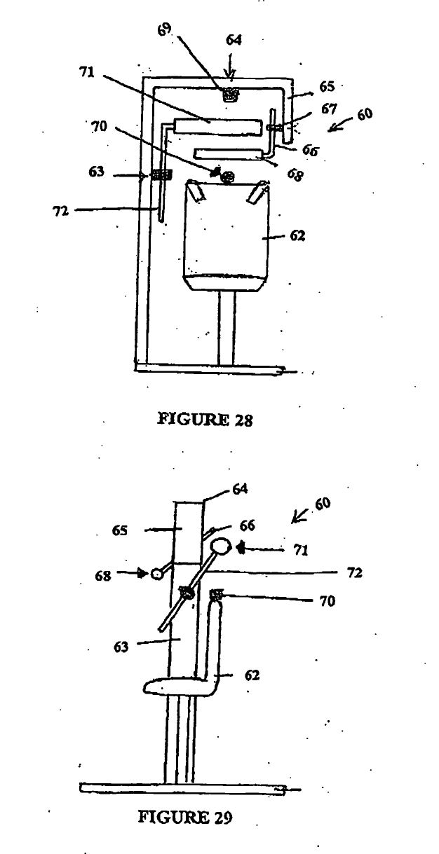

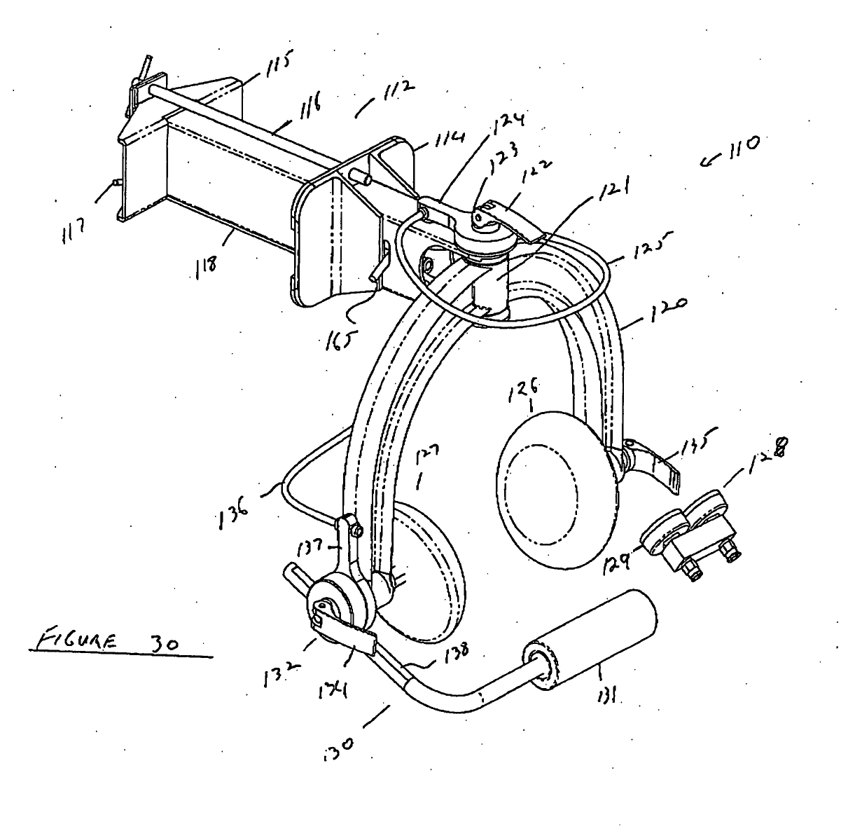

An alternative embodiment is shown in FIGS. 28 and 29 in which a subject is seated during testing. This arrangement has the advantage of requiring less space and may therefore be more suited to location in professional rooms. This embodiment may also have the capacity via a mechanical pivot system to be adjusted so testing may be performed in the horizontal position if desired.

The apparatus 60 is formed by a base 73 supporting a chair 62 and upright post 63. The upright post 63 is continuos with a horizontal bar 64 and downward arm 65.

The rotatable lever arm 66 is supported on a hub 67 which receives the lever arm. The lever arm 66 may be increased or decreased in its functional length by sliding movement relative to the hub 67 so that a subject contacting arm 68 is moved towards or away from the hub. The lever arm 66 and subject contacting arm 68 may be static or rotatable in operation. Rotation may be controlled by a motor or a resistance load such as a suspended variable weight or a pneumatic cylinder arrangement as described below. The lever arm 66 as positioned is suitable for assessing performance in the sagittal plane. The lever arm 66 may be removed from the hub 67 and positioned in second hub 69 for positioning to assess muscle functional when rotating in the transverse plane. Similarly the lever arm 66 may be positioned in third hub 70 for use to assess lateral flexion which occurs predominantly in the coronal plane. The first hub 63 may be suitable for alignment with the anatomical feature of the C7/T1 joint which is located on or around the AOR of the head and neck flexion and extension action. The lever arm 72 and the subject contact arm 71 can be fixed in the hub 63 for monitoring of head motion during C-C muscle testing but additionally may be used to assess large cervical muscles in sagittal plane flexion and extension. The hub 69 may be suitable for alignment with the AOR of the cranio-cervical muscles through the C₁/C₂ joint. The hub 67 may be suitable for alignment with axis of rotation for C-C flexion extension through the C₀/C₁ joint. All the hubs may be adjustable for axis position, lever arm length and lever arm angle. This embodiment therefore has the capacity to measure muscle performance of both C-C and Cervical muscle groups in all directions eg. Flexion, extension, axial rotation, lateral flexion, additional to monitoring head force when performing C-C muscle tests.

The subject contact arm 71 and lever arm 72 permit an assessment of pressure backwards or forwards, as provided by the rear of a subject's head. A significant increase or decrease in such pressure may be an indication of inappropriate technique in performance of a movement by the subject. Of course, further extensions and accessories as described earlier may be applied to the apparatus. For example, electronic force detecting means such as load cells may be used to determine force. Data may be electronically provided to a processing device such as a computer to determine torque. A visual or audible cue may be provided to a subject or clinician to indicate a level of performance during a prescribed movement or exercise. An LED or VDU display may be particularly suitable.

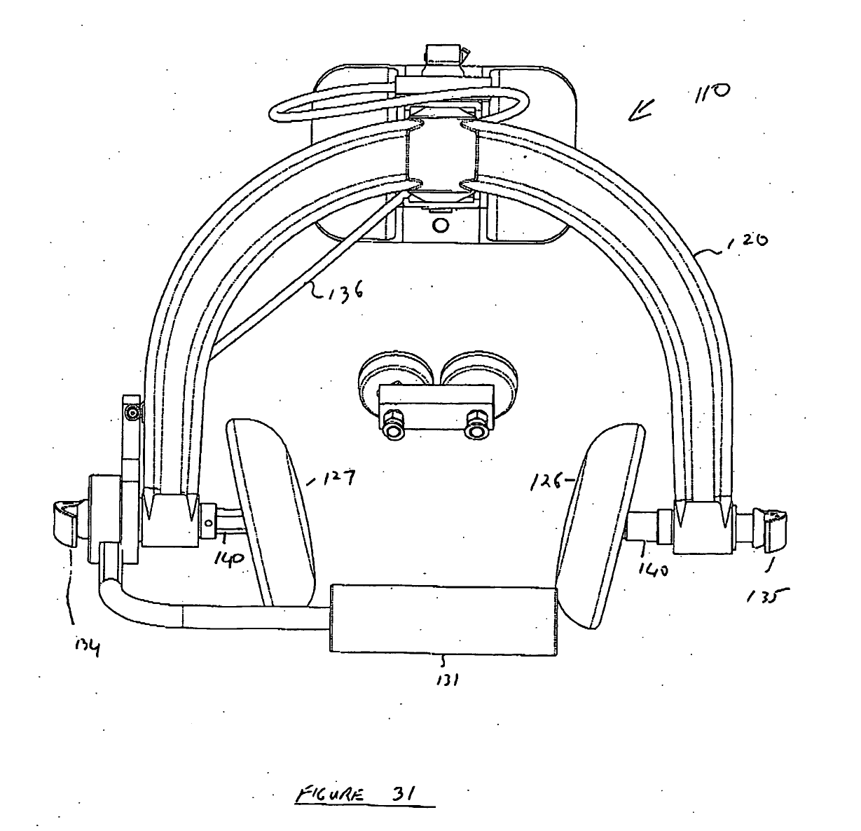

FIG. 30 shows an embodiment of an apparatus or device of the present invention 110 having mounting means in the form of adjustable clamp 112 with paired spaced jaws 114, 115. The jaws may be advanced towards or retracted from each other by rotatable threaded shaft 116 and its paired spaced shaft 117. The clamp 112 is ideally suited for mounting on an upright structure such as a post or, preferably, the edge of a door such as a door jamb. Having the shaft area 118 central allows for the clamp to be fitted on either a left- or right-hard side of the door as required. The shaft extends through to pivotally mount a support frame 120 which is formed in a U-shape and dimensioned to fit over a subject's head from side to side. The support frame 120 is pivotally mounted to the shat 118 by a pivot joint 121. Brake means is supplied to the pivoted joint in the form of a quick release lever 122 with a cammed end 123 which, in operation, compresses a deformable shaft to lock on a bore in which it is located. An arm 124 is engaged with the support frame 120 and is, in turn, connected to bowden cable 125. The connection is such as to allow transfer of movement to the bowden cable in both clockwise and anticlockwise rotation.

Locating means in the form of paired ear pieces 126, 127 are fitted to the lower ends of the support frame 120 and for positioning on a subject's ears. The support frame 120 may act as a lever arm for rotation of subject's head in the transverse plane. Positioning of the axis of rotation of the support frame roughly coincident with the axis of rotation of the subject's head (ie. around C₁/C₂ axis) will provide an accurate or a reasonably accurate indication of torque produced by operation of the relevant muscles. This device may be used to assess the C-C rotators and may also be used for assessment of the larger cervical muscle rotators in a more gross movement. In operation, the quick release lever 122 is placed into position to allow rotation of the support frame 120. The apparatus may be adjusted for isometric of isokinetic activity. The subject will then perform a prescribed exercise which may provide a visual output in one of the gauges 128, 129 which are not shown connected to hydraulic lines with this view. Alternatively, resistance to movement might be provided through the mean described further below so that a patient may conduct isokinetic exercises against a substantially constant resistance. A second lever arm 130 is formed with a padded chin section 131. The lever arm 130 is mounted at a point 132 roughly coincident with an axis between the centres of the ear pads 126, 127. The ear pads are mounted for rotational movement relative to the support frame 120 and to act as the head application point of resistance durial axial rotation motion. Quick release levers 134, 135 are provided to allow the ear pads to be moved in or outwards for adjustment to a subject's head width. The lever arm 130 is also releasably mounted at the point 132 and may be adjusted to conform to a patient's anatomy. A second bowden cable 136 is attached to an indicator arm 137. The lever arm 130 may be recessed 138 to resist rotation of the lever arm relative to its mounting point. The lever arm may also be calibrated to indicate the length from the point of rotation to the point of application of force at the chin pad 131. This measurement may also be automatic by use of an appropriate electronic arrangement, preferably using potentiometers. The torque produced may be established by multiplying the length of the arm 137 by the force produced and shown in the gauge 128, 125. Alternatively, the gauges may be calibrated to provide a direct indication of torque.

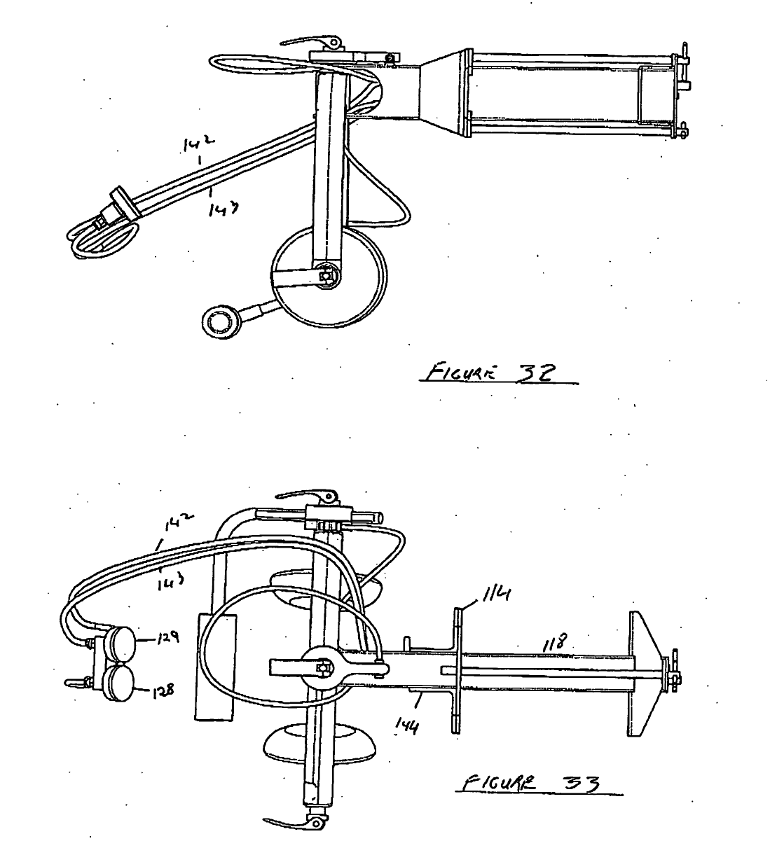

FIG. 31 is a front view of the same arrangement of an apparatus 110 highlighting the shafts 140 provided a sliding adjustment of the ear pads 126, 127 after release of the levers 134, 135.

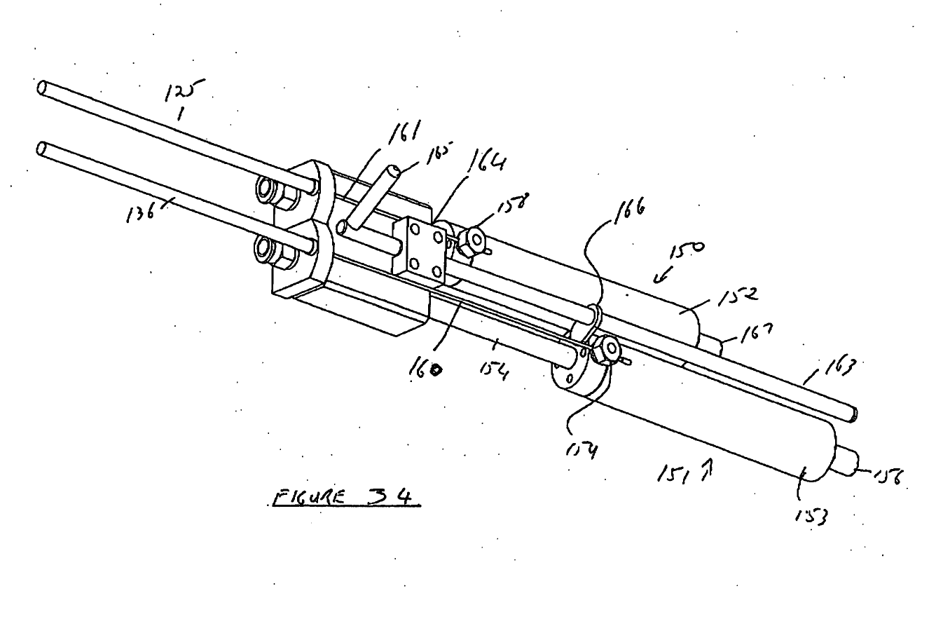

FIG. 32 is a side view of the embodiment of FIG. 30 in which hydraulic lines 142, 143 are in fluid connection the gauges 128, 129 as is also apparent in top view in FIG. 33. The latter view also shows the jaw 114 slidably mounted to the shaft 118 by bracket 144.

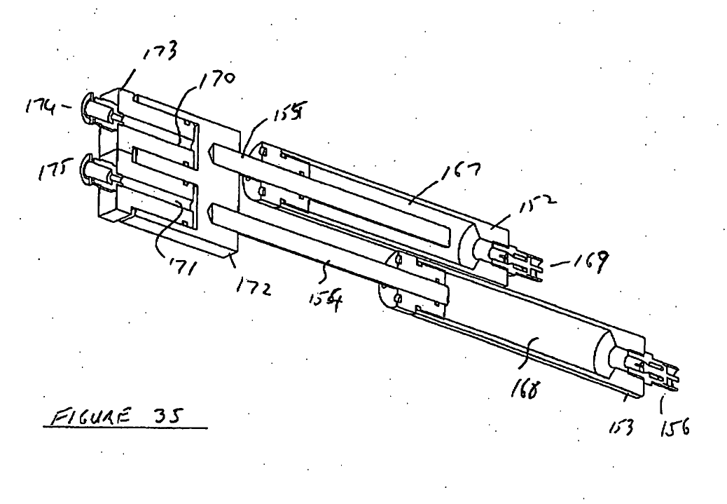

FIG. 34 shows two piston arrangements 150, 151 comprising an outer cylinder 152, 153, respectively, and shafts of which only the closest 154 is visible. The shaft 154 is slidably mounted in the outer cylinder 153 which has a valve arrangement 156 to allow alteration of internal pressure in the cylinder. In a preferred embodiment, the cylinder is gas filled and the valve arrangement 156 may be used for increasing or decreasing pressure therein. Bowden cables 125, 136 are engaged with respective fixing screws 158, 159 by the inner slidable core 160. Lockout actuator 163 is rotatably mounted in a block 164 with an external handle 165 provided to extrude through the shaft 118 as seen in FIG. 30. Rotation of the handle 165 causes a finger 166 to rotate into abutting contact with a top of the outer cylinder 153, thereby bridging the shaft 154 and preventing it from sliding inwardly into the cylinder. This position may be suitable for isometric contraction. At the same time, the shaft 155 (not seen) of cylinder 152 is able to slidably penetrate into the cylinder.

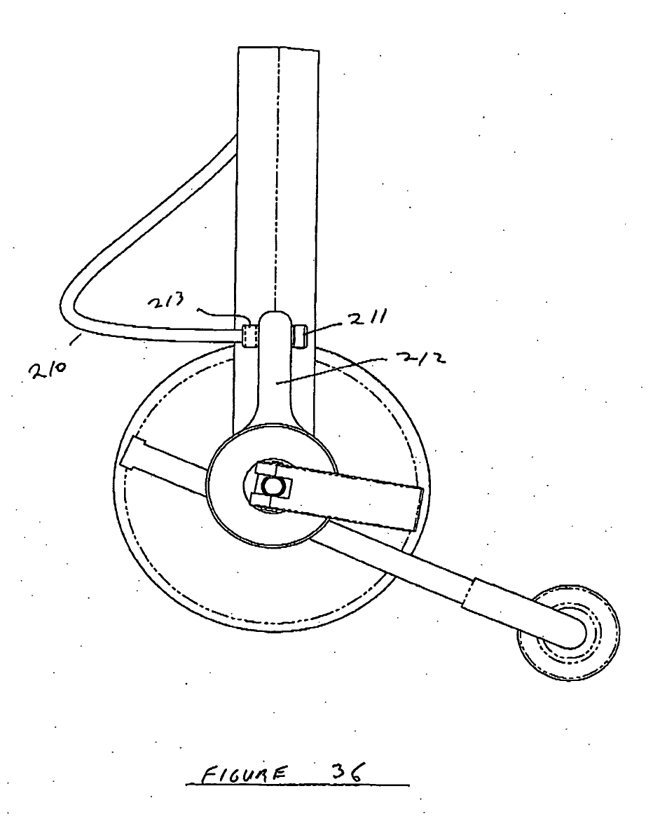

A sectional view in FIG. 35 shows the features described in more detail. The shafts are relatively small in comparison to the cavities 167, 168 in the outer cylinders 152, 153. Movement of the shafts into the cylinders therefore does not cause any substantial increase in pressure within the cylinders by displacement of gas. Movement of the pistons is resisted by a force equal to pressure by the surface area of the end of the piston. The valves 156, 169 are also apparent allowing for variation of the pressure within the cavities 167, 168. The ends of the shafts 154, 155 remote from the cylinders are co-operatively located to compress hydraulic fluid located in chambers 170, 171 by movement of a piston 172 against ahead 173. The chambers 170, 171 are in fluid connection with the gauges 128, 129, respectively. A bleed valve 174, 175 is provided for each chamber and may be used to remove air in the lines.

In operation, a maximal voluntray contraction (MVC) may be recorded. A suitable percentage of the MVC may be calculated and the appropriate pressure for the cylinder provided so that pressure×surface area of the piston 172 will result in the chosen resistance. In one embodiment, the area of the end of shaft 154 may be formed in a preferred relationship to the area of piston 172 for compressing hydraulic fluid. For example, if the area is formed as 20% of the piston 172, a resistance force of 20% of the MVC may be obtained by simply equalising pressure in the cylinder 168 to the MVC reading.

Consistent resistance may be applied throughout range.

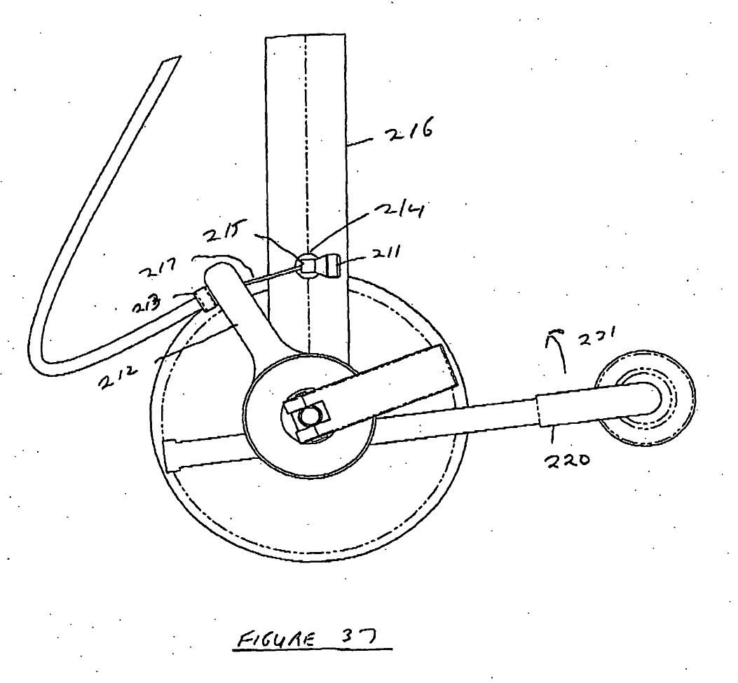

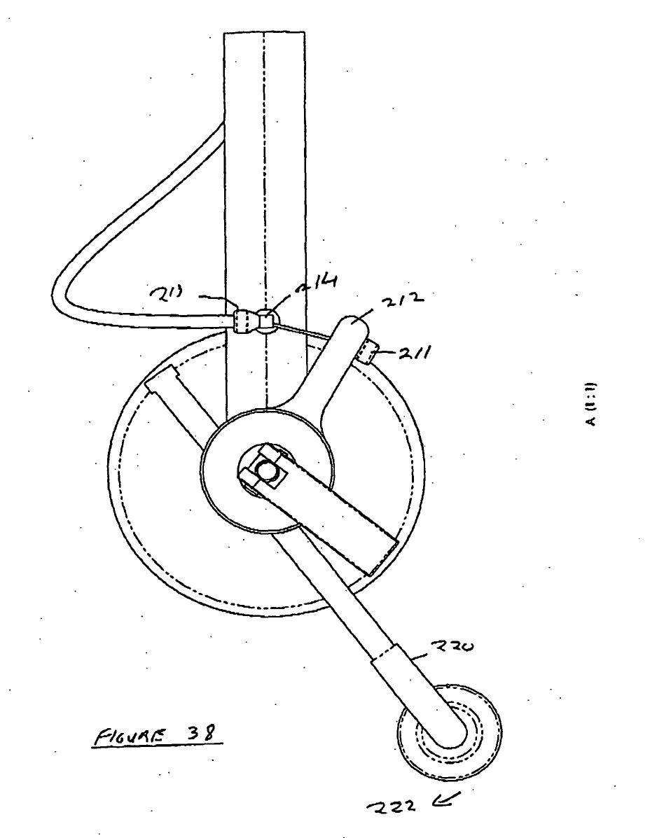

The present invention also uses a connection as shown in FIGS. 36 to 38 for bilateral use of a bowden cable. FIG. 36 shows an arrangement in which a bowden cable 210 terminates in an end cap 211 situated in contact with the mounting or indicator arm 212. A end ring 213 is provided proximally to the mounting arm 212. FIG. 37 shows a stud 214 with an eye 215 fitted to support member 216. The centre cord 217 passes through the eye 215 on to end cap 211. Rotation of the lever arm 220 in the direction of arrow 221 causes the indicator arm 212 to move relative to the end cap 211 and stud 214, thereby displacing the end ring 213 and pressurising the centre cord 217 which will register on a gauge or, alternatively, cause the piston described above to move into the cylinder. The indicator arm 212 has a recess open medially sufficiently to locate and pass over the stud 214 and eye 215. FIG. 38 shows the arm 220 moving in the direction of opposite arrow 222 which results in the end ring 213 engaging the eye 214 and indicator arm 212 and end cap 211 are displaced to also tension the cable and transfer force. The preset arrangement therefore allows for both extension and flexion to be measurer by the same components, thereby providing great utility in the present device.

Assessment of performance may be based on a subject's ability to maintain a steady torque within preset limits which may, for example, be 1%, 3%, 5%, 10%, or 20% either side of the prescribed level. Performance may be rated by accuracy within the set margins, frequency of excursion beyond the margins and/or wavelet analysis (generally “steadiness of performance”).

The emphasis in the preferred embodiment has been on cranio-cervical muscle monitoring. However, it is clear that the apparatus may be used to assess the function of larger muscle masses. The method may then extend to also assessing the function of a larger muscle mass such as the cervical flexors, extensors, lateral flexors, and rotators (“the larger cervical muscle”). This assessment may include positioning the AOP of the lever arm at or around the axis of rotation of the anatomical strut activated by the larger muscle group.

Subsequent analysis may extend to comparisons of the cranio-cervical muscle performance in different planes (eg. sagittal, coronal and transverse). Comparisons may be in the form of ratios. For example C-C Flexion: C-C Extension, Left C-C axial rotation: Right C-C axial rotation.

The analysis may further comprise a comparison of the cranio-cervical muscle functions with that of the larger cervical muscle groups. Comparisons may be in the form of ratios. For example: C-C flexion: Cervical flexion, C-C extension: Cervical extension, C-C flexion: cervical extension. For example, a matrix may be formed as follows:

| | | | C-C | C-C | C-C | C-C |
|---|---|---|---|---|---|---|
| | | | right | left | right | left |
| | C-C | C-C | axial | axial | lateral | lateral |
| | Flexion | Extension | rotation | rotation | flexion | flexion. |
| Cervical | X₁ | X₂ | X₃ | X₄ | X₅ | X₆ |
| flexion | | | | | | |
| Cervical | X₇ | X₈ | X₉ | X₁₀ | X₁₁ | X₁₂ |
| extension | | | | | | |
| Cervical | X₁₃ | X₁₄ | X₁₅ | X₁₆ | X₁₇ | X₁₈ |
| right axial | | | | | | |
| rotation | | | | | | |
| Cervical | X₁₉ | X₂₀ | X₂₁ | X₂₂ | X₂₃ | X₂₄ |
| left axial | | | | | | |
| rotation | | | | | | |
| Cervical | X₂₅ | X₂₆ | X₂₇ | X₂₈ | X₂₉ | X₃₀ |
| right lateral | | | | | | |
| flexion | | | | | | |
| Cervical | X₃₁ | X₃₂ | X₃₃ | X₃₄ | X₃₅ | X₃₆ |
| left lateral | | | | | | |
| flexion | | | | | | |

wherein X₁-X₃₆ are indices which may provide an indication of relative function. Any 1 or more of the indices may be calculated as considered appropriate. The indices may be calculated by any useful means such as division of one torque calculation for a muscle group into the torque calculation of the associated muscle group. Alternatively, addition, subtraction or multiplication may be found useful.

The results may be compared with and added to a data base to provide an ongoing development and scope of the data base. The results can be used to formulate specific tailored exercise programs and later used following rehabilitation to monitor progress and advance exercise. Therefore this system facilitates a method of diagnosis and rehabilitation of all neck muscles. Rehabilitation can be performed on either the clinical or the home version of the dynamometer. This method is also applicable to other muscle groups in the body which are interrelated to each other.

The “take home” versions of the present invention may be provided. A clinician may then provide a patient with access, temporary or permanent, to an apparatus. The patient may then self monitor during a rehabilitation/exercise programme to ensure maximum therapeutic advantage from the performance of prescribed activities.

A preferred embodiment of the present apparatus includes the ability to measure the torque of both the cranio-cervical muscles and the cervical muscles:

(1). Torque of the cranio-cervical muscles is measured about an axis of rotation located at the cranio-cervical junction as described previously;

(2) Torque of the cervical muscles is measured about an axis of rotation located at the cervico-thoracic junction.

This leads to a novel method of assessment of neck muscle function in that the relationship between the performance of the cranio-cervical and cervical muscles may be assessed in the same individual. Therefore, such performance factors as strength and endurance ratio's between the cranio-cervical and cervical muscle groups for, eg. the flexor muscles may be determined. This has not been used previously. The ratio of cranio-cervical:cervical ratio performance may of considerable use.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appendant claims.

### Glossary of Terms

Cranio-Cervical Terminology

Cranio-Cervical (C-C) Region—is that region of the spine where the skull joins onto the upper cervical spine and its associated soft tissue attachments.

C-C Motion—describes motion of the head on the neck and is primarily produced and controlled by the muscles that attach the spine directly to the skull or jaw. The principle anatomical directions of C-C spine motion are C-C flexion, extension, axial rotation, and lateral flexion however infinite combinations of these movements may occur.

C-C Flexion—involves a specific roiling action of the head on the neck resulting in a nodding type movement and an approximation of the chin to the front of the neck. This motion is primarily produced by a group of muscles called the C-C Flexor Muscles.

C-C Extension—involves a specific roiling action of the head on the neck resulting in a nodding type movement and a separation of the chin from the front of the neck. This motion is primarily produced by a group of muscles called the C-C Extensor Muscles.

C-C Axial Rotation—involves a specific rotation action of the head on the neck resulting in a “NO” type motion of the head on the neck. This motion is primarily produced by a group of muscles called the C-C Axial Rotator Muscles.

C-C Lateral Flexion—involves a specific slant of the head on the neck such that the ear leans toward the shoulder. This motion is primarily produced by a group of muscles called the C-C Lateral Flexor Muscles.

Cervical Terminology

Cervical Motion—describes motion of the head and the neck together on the thorax and is primarily produced and controlled by the muscles that attach the cervical spine and the head to the thorax. The principle anatomical directions of Cervical spine motion are Cervical flexion, extension, axial rotation, and lateral flexion however infinite combinations of these movements may occur.

Cervical Flexion—involves an anterior motion of the head and cervical spine together in the sagittal plane and an approximation of the chin to the chest. This motion is primarily produced by a group of muscles called the Cervical Flexor Muscles.

Cervical Extension—involves a posterior motion of the head and cervical spine together in the sagittal plane and an approximation of the back of the head to the thorax. This motion is primarily produced by a group of muscles called the Cervical Extensor Muscles.

Cervical Axial Rotation—involves a specific rotation action of the head and neck together on the thorax in the transverse plane. This motion is primarily produced by a group of muscles called the Cervical Axial Rotator Muscles.

Cervical Lateral Flexion—involves motion of the head and neck together on the thorax in the coronal plane such that the ear leans toward the shoulder. This motion is primarily produced by a group of muscles called the Cervical Lateral Flexor Muscles.

Muscle Performance Terminology

Dynamometer—an instrument for measuring the force of muscular contraction.

Muscle Strength—Maximal force that can be grated by a muscle or muscle group (unit: Newtons).

Force—That which changes or tends to change the state of rest or motion in matter (unit: newton)

Torque—Effectiveness of a force to produce axial rotation (unit: newton-meter). Calculated by multiplying force by distance from the axis of rotation.

Axis of Rotation—When a rigid body, in this case the head, moves in one plane along an arc there is a point in that body that does not move (ie. has zero velocity). This point represents the center of rotation. An axis through this center of rotation, perpendicular to the plane of motion is the axis of rotation.

Static muscle torque—Torque generated by a muscle(s) without any motion occurring about the axis of rotation. The motion is fully opposed and the muscle contraction results in no movement. This is also termed an isometric muscle contraction.

Dynamic muscle torque—Effectiveness of a muscle (s) to produce axial rotation. In a dynamic muscle contraction a muscle may shorten (concentric) or lengthen (eccentric). This is also termed an isotonic muscle contraction.

Maximal voluntary contraction—Maximal torque a muscle or group of muscles can exert in one single maxim exertion intensity.

Muscular endurance—The time limit of a persons ability to maintain either a specific isometric force or a specific power level involving combinations of concentric or eccentric muscular contractions.

Transducer—Is a device that produces a voltage proportional to the quantity to be measure eg. muscle force

Load Cell—Is a transducer involving a metal object with strain gauges attached to it which give a voltage output proportional to the applied force.

Exercise intensity—A specific level of maintenance of muscular activity that can be quantified in terms of power (energy expenditure or work performed per unit of time), isometric force sustained, or velocity of progression.

### REFERENCES

1. Bovim, G., R. Berg, and L. G. Dale, Cervicogenic Headache: anaesthetic blockades of cervical nerves (C2-C5) and facet joint (C2/3). Pain, 1992. 49: p. 315-320.
2. Cote, P., J. D. Cassidy, and L. Carroll, The saskatchewan health and back pain survey: The prevalence of neck pain and related disability in Saskatchewan adults. Spine; 1998. 23(15): p. 1689-1698.
3. Makela, M., et al., Prevalence, determinants, and consequences of chronic neck pain in Finland. Am J Epidemiol, 1991. 134: p. 1356-67.
4. MAIC. 2002: Brisbane.
5. Rasmussen, B. K., et al., Epidemiology of headache in a general population—a prevalence study. Journal of Clinical Epidemiology, 1991. 44: p. 1147-1157.
6. Pfaffenrath V. and H. Kaube, Diagnostics of cervicogenic headache. Funet Neurol, 1990. 5: p. 159-64.
7. Nilsson, N., The prevalence of cervicogenic headache in a random population sample of 20-59 year olds. Spine, 1995. 20: p. 1884-1888.
8. PABC, PABC Market Research. 2001, Physiotherapy Association of British Columbia (PABC@bcphysio.org).
9. Falla, D., G. Jull, and P. Hodges, Neck pain patients demonstrate reduced activity of the deep neck flexor muscles during performance of the craniocervical flexion test. Submitted for publication, 2003.
10. Jull, G., C. Barrett, and R. Magee, Further characterization muscle dysfunction in cervical headache. Cephalalgia, 1999. 19: p. 179-185.
11. Jull G. A., Deep cervical flexor muscle dysfunction in whiplash. Journal of Musculoskeletal Pain, 2000. 8: p. 143-154.
12. Jull, G., et al., A randomised controlled trial of exercise and Manipulative therapy for cervicogenic headache. Spine, 2002. 27(17): p. 1835-1843.
13. Watson, D. H. and P. H. Trott, Cervical headache: an investigation of natural head posture and upper cervical flexor muscle performance. Cephalalgia, 1993. 13: p. 272-284.
14. Andrey, M. E., et al., Neurogenic atrophy of suboccipital muscles after a cervical injury. American Journal of Physical Medicine and Rehabilitation, 1998. 77: p. 545-549.
15. Hallgren, R. C., P. B. Greenman, and J. J. Rechtien, Atrophy of the suboccipital muscles in patients with chronic pain: a pilot study. JAOA, 1994. 94(12): p. 1032-1038.
16. McPartland, J. M., R. R. Brodeur, and R. C. Hallgren, Chronic neck pain, standing balance, and suboccipital muscle atrophy—a pilot study. Journal of Manipulative and Physiological Therapeutics, 1997. 20(1); p. 24-29.
17. Uhlig, Y., B. R. Weber, and D. G. M. Muntener, Fiber composition and fiber transformations in neck muscles of patients with dysfunction of the cervical spine. Journal of Orthopaedic Research. 1995. 13: p. 240-249.
18. Treleaven, J., G. Jull, and M. Sterling, Dizziness and unsteadiness following whiplash injury—characteristic features and relationship to cervical joint position error. Journal of Rehabilitative Medicine, 2003. 34: p. 1-8.
19. Tjell, C. and U. Rosenhall, Smooth pursuit neck torsion test: A specific test for cervical dizziness. The American Journal of Otology, 1998. 19: p. 76-81.
20. Revel, M., C. Andre-Deshays, and M. Minguet, Cervcocephalic kinesthetic sensibility in patients with cervical pain. Archives of Physical Medicine and Rehabilitation, 1991. 72: p. 288-91.
21. Falla, D., et al., Myoelectric manifestations of sternocleidomastoid and anterior scalene muscle fatigue in chronic neck pain patients. Clinical Neurophysiology, 2003. 114(3): p. 488-495.
22 Grimmer, K. and P. Trott, The association between cervical excursion on angles and cervical short flexor muscle endurance. Australian Journal of Physiotherapy, 1998. 44: p. 201-207.
23. Falla, D., et al., Neuromuscular efficiency of the sternocleidomastoid and anterior scalene muscles in patients with neck pain. Submitted for publication, 2003.
24. Falla, D., et al., Electromyographic analysis of the deep cervical flexor muscles during the craniocervical flexion test. Submitted for publication, 2003.
25. Norton, K and T. Olds. Anthropometrica. 1996. Sydney: University of New South Wales Press;
26. Amevo, B., et al., Instantaneous of rotation of the typical cervical motion segments: 1. an empiracal study of technical errors. Clinical Biomechanics, 1991. 6: p. 31-37.
27. Panjabi M. M. and A. A. White, Biomechanics in the Musculoskeletal System. 2001, New York: Churchill Livingstone.
28. Reuleaux F., The kinematics of machinery: Outline of a theory of machines (translated by Kennedy, A. B. W.). 1875, New York: Dover. 56-70.
29. Worth, D. R. and G. Selvik, Movements of the cranio-vertebral joints, in Modern Manual Therapy, G. P. Grieve, Editor. 1986, Churchill Livingstone: Edinburgh.
30. vanMameran, H., et al. Cervical spine motion in the sagittal plane II: Position of segmental averaged instantaneous center of rotation—A cineradiographic study. Spine, 1992. 17(5): p. 467-474.
31. Harms-Ringdahl, K, et al., Load moments and myoelectric activity when the cervical spine is held in full flexion and extension. Ergonomics, 1986. 29(12): p. 1539-1552.

1. An apparatus for assessing and/or exercising cranio-cervical musculature in a subject, the apparatus comprising:
a force receiving member having a subject input region adapted to receive force input from the head of the subject;
an axis of rotation of the force receiving member spaced from the subject input region and substantially aligned with the C0/1 joint of the subject; and
torque assessment means for determining or assessing torque produced at the axis of rotation of the force receiving member.

2-111. (canceled)

112. The apparatus of claim 1, wherein the apparatus is adapted to assess performance of the cranio-cervical muscles in one or more of flexion, extension, axial rotation, lateral flexion and a combination or combinations thereof.

113. The apparatus of claim 1, wherein the force receiving member is a lever arm of any suitable shape, having an arm portion and an offset subject engaging portion, the subject engaging portion forming or including the subject input region.

114. The apparatus of claim 113, further comprising a support frame and locking means for fixing the lever arm to the support frame to facilitate measurement or assessment of torque, the locking means adapted to permit variation of the perpendicular distance and angle of the lever arm.

115. The apparatus of claim 114, wherein the support frame includes a frame member adapted for location around a subject's head.

116. The apparatus of claim 114, wherein the support frame includes ear pads for location on either side of the subject's head to position an axis of rotation of the lever arm relative to the subject.

117. The apparatus of claim 116, wherein the ear pads are rotatably mounted to the frame member to allow rotation of the subject's head relative to the frame member in a sagittal plane.

118. The apparatus of claim 116, wherein the lever arm is adapted for positioning at or around a subject's chin and is pivoted on an axis between the ear pads which are adapted to align the axis of rotation of the lever arm with or around the subject's axis of rotation when conducting C-C flexion and extension.

119. The apparatus of claim 115, wherein the frame member is superiorly pivoted to form a second lever arm in the apparatus and to permit rotation of a subject's head in the transverse plane.

120. The apparatus of claim 1, further comprising mounting means for fixing to a support structure.

121. The apparatus of claim 120, further comprising travel control means to provide controlled rotation of one or both lever arms.

122. The apparatus of claim 121, wherein the travel control means comprises one or more piston arrangements connected directly or indirectly to a corresponding lever arm, each piston arrangement having a piston mounted slidably in a cylinder, the cylinder containing a fluid which may be pressurized to resist inward movement of the piston.

123. The apparatus of claim 122, having two piston arrangements, one adapted for flexion/extension of the C-C musculature and the other adapted for rotation of the C-C musculature.

124. The apparatus of claim 123, further comprising a third piston arrangement provided for lateral C-C flexion and including a mounting for a third lever arm positioned for assessing lateral flexion.

125. The apparatus of claim 123, further comprising a switch arrangement to prevent operation of one or other of the pistons at any one time.

126. The apparatus of claim 113, further comprising adjustment means for altering the position of the axis of rotation of the lever arm relative to the base.

127. The apparatus of claim 126, wherein the adjustment means is adapted to orientate the lever arm for operation in the sagittal, coronal and transverse planes.

128. The apparatus of claim 1, further comprising subject head support means adapted to support the head of a subject during assessment.

129. The apparatus of claim 128, wherein the head support means is slidable with low friction.

130. The apparatus of claim 1 also comprising electromyographic monitoring means for monitoring the electrical activity of muscles under review.

131. A dynamometer for assessing muscular generation of torque by C-C muscles of a subject, the dynamometer comprising:
one or more lever arms rotatably mounted to a support frame and having a subject contact region;
torque measuring or assessing means for measuring or assessing torque produced by the subject; and
locking means for locking the lever arm in position relative to an axis of rotation of the lever arm substantially in alignment with the C0/1 joint of the subject;
wherein the length of the lever arm and its angle are variable.

132. The dynamometer of claim 131, further comprising adjustment means for adjusting the axis of rotation of the lever arm relative to the support frame and/or a base of the dynamometer.

133. The dynamometer of claim 132, wherein the adjustment means comprises two or more mounting positions for the lever arm to be alternatively mounted thereon for assessing the function of C-C muscles in sagittal, coronal and transverse planes.

134. The dynamometer of claim 131, wherein the support frame comprises a U-shaped support member having ear pads which are adjustable inwardly and outwardly relative to each other and adapted for location over the ears of a subject.

135. The dynamometer of claim 131, wherein the axis of rotation of a lever arm for assessing C-C muscle function in flexion/extension is approximately coincident with the anatomical axis of rotation of the subject.

136. The dynamometer of claim 134, wherein the U-shaped member is pivotally engaged to a shaft and variable between a locked and rotatable engagement with the shaft and adapted to act as a lever arm in axial rotation in the transverse plane.

137. A method of assessing and/or exercising C-C musculature, the method comprising:
conducting a repeated performance of C-C muscle activity including flexion/extension about the C0/1 joint, and optionally axial rotation and lateral flexion; and
monitoring performance of the muscle groups during the C-C muscle activity.

138. The method of claim 137, wherein the muscle activity includes maximal voluntary contraction and/or sustained voluntary contraction and/or repeated oscillatory voluntary contractions when conducting one or more of flexion/extension, axial rotation and lateral rotation or a combination thereof.

139. An apparatus for improving and/or assessing performance of cranio-cervical muscles, the apparatus comprising:
an adjustable clamp for fixing the apparatus to a support structure;
a shaft supporting the clamp;
a support frame member adapted for location over a subject's head, the support frame member rotatably fixed to the shaft and adapted to act as a lever arm for axial rotation;
brake means for locking and releasing the support frame member to rotate;
ear pads rotatably fixed to the support frame member and adapted to locate on or over a subject's ears, a space between the ear pads being adjustable; and
a lever arm extending from the support frame and adapted to engage a subject for flexion extension assessment;
wherein the lever arm is adjustable in both length and a direction of extension from the support frame member and wherein the ear pieces are positioned to substantially align an axis of rotation of the lever arm and approximate anatomical axis of rotation of the subject during flexion/extension.

140. The apparatus of claim 139, further comprising an alternative mounting point for the frame member, wherein the alternative mounting point is located to align an axis of rotation of the frame member with an approximate anatomical axis of rotation of the subject during lateral flexion.
